SEQUENCE LISTING (2 sequences)

SEQ ID NO: 1 - DNA, 18 bp, synthetic construct
  misc_feature 1..18: note = GABRA3 forward primer
  source 1..18: mol_type = other DNA
gaccacgcccaacaagct

SEQ ID NO: 2 - DNA, 29 bp, synthetic construct
  misc_feature 1..29: note = GABRA3 reverse primer
  source 1..29: mol_type = other DNA
agcatgaattgttaacctcattgtataga

(19) United States
(12) Patent Application Publication
(10) Pub. No.: US 2026/0062760 A1
(43) Pub. Date: Mar. 5, 2026

(54) METHODS OF TREATMENT AND DIAGNOSIS OF MULTIPLE MYELOMA PROGRESSION

(71) Applicant: Morehouse School of Medicine, Atlanta, GA (US)

(72) Inventors: James W. LILLARD, JR., Smyrna, GA (US); Olayinka ADEBAYO, Douglasville, GA (US)

(21) Appl. No.: 19/353,122

(22) Filed: Oct. 8, 2025

Related U.S. Application Data
(62) Division of application No. 17/659,946, filed on Apr. 20, 2022.

(51) Int. Cl. C12Q 1/6886; A61K 31/713; A61P 35/04

(52) U.S. Cl. CPC C12Q 1/6886; A61K 31/713; A61P 35/04; C12Q 2600/112; C12Q 2600/158

(57) ABSTRACT

A method of treating multiple myeloma, comprising administering one or more agents increasing or inhibiting the expression or activity of one or more MM biomarkers to inhibit the progression of multiple myeloma, wherein the one or more MM biomarkers correspond to gene products from one or more of GABRA3, CTAG2, MAGEA6, SOHLH1, MAGEA1, AFAP1-AS1; CBX2, LINC00484, KIF7, TMSB15A, NEK2, NTRK1, CCND2, NES, PKP2, C1orf226, IFITM1, CDH23, AGRN, DHX58, and LINC02576. A method of diagnosing and treating multiple myeloma in a subject comprises, (a) measuring the level of one or more biomarkers in a sample; (b) comparing the level of the one or more biomarkers to a reference level of the one or more biomarkers; (c) making a diagnosis based on the result of the comparing step; and (d) treating the subject with one or more active agents where the subject is diagnosed with multiple myeloma.

This application is a Divisional of U.S. application Ser. No. 17/659,946, filed Apr. 20, 2022. The entirety of the aforementioned applications is incorporated herein by reference.

This invention was made with government support under U54CA118638 awarded by the National Institute of Health and the Ruth L. Kirschstein National Research Service Award (NRSA) (T32). The government has certain rights in the invention.

### FIELD

This application generally relates to the field of treatments for the inhibition or prevention of the growth and/or migration of cancer cells, in particular, multiple myeloma.

### BACKGROUND

The American Cancer Society estimates that in 2021 approximately 34,920 new cases of Multiple Myeloma (MM) will be diagnosed, and approximately 12,410 deaths are expected to occur. Despite the progress made for treating MM in the past 40 years, including introducing new immunomodulatory drugs and proteasome inhibitors, it is still an incurable disease. According to current data, the five-year survival rate is 52%. MM is a heterogeneous disease with a diverse clinical course expressed by differences in therapeutic strategies' effectiveness and ability to develop chemoresistance.

MM is characterized by malignant proliferation of plasma cells and is defined as incurable hematological malignancies that are pathologically linked with aberrant NF-xB activation. MM remains an incurable disease with unpredictable refractory mechanisms. Despite therapeutic advances that have increased the median overall survival rate to over 3-fold in the last fifteen years, chemoresistance is a major hurdle in the treatment of multiple myeloma. Therefore, a better understanding of the molecular mechanism of chemoresistance in newly diagnosed MM on standard therapies is needed.

The molecular mechanisms underlying chemoresistance in newly diagnosed multiple myeloma (MM) patients receiving standard therapies (lenalidomide, bortezomib, and dexamethasone) are poorly understood and are often ineffective. The identification of clinically relevant gene networks associated with deaths due to MM can uncover novel mechanisms, drug targets, and prognostic biomarkers to improve the treatment of the disease.

### SUMMARY

In one aspect, a method of diagnosing and treating multiple myeloma in a subject comprises: (a) measuring the level of one or more biomarkers in a sample from the subject; (b) comparing the level of the one or more biomarkers to a reference level of the one or more biomarkers; (c) making a diagnosis based on the result of the comparing step; and (d) treating the subject with one or more active agents where the subject is diagnosed with multiple myeloma.

In another aspect, method for determining disease progression or risk for metastasis in a subject with multiple myeloma, comprises the steps of: (a) measuring the level of one or more biomarkers in a first sample obtained from the subject with multiple myeloma at a first time point; (b) measuring the level of the one or more biomarkers in a second sample obtained from the subject at a second time point; (c) comparing the level of the one or more biomarkers at the first time point to the level of the one or more biomarkers at the second time point; (d) determining the disease progression between the first and the second time point based on the result of step (c); and (e) further treating the subject with one or more active agents if the multiple myeloma has progressed.

In another aspect, a method for determining the efficacy of a treatment for multiple myeloma in a subject, comprises the steps of: (a) measuring the level of one or more biomarkers in a first sample obtained from the subject at a first time point; (b) measuring the level of the one or more biomarkers in a second sample obtained from the subject at a second time point, wherein the subject is under treatment at the second time point; (c) comparing the level of the one or more biomarkers at the first time point to the level of the one or more biomarkers at the second time point; (d) determining the efficacy of the treatment based on the result of step (c); and (e) further treating the subject with one or more active agents if the efficacy has been found to be insufficient for treatment. In certain preferred embodiments, the one or more active agents in step (e) include one or more active agents that were not administered in the previous treatment.

In another aspect, a method of treating multiple myeloma, comprises administering to a subject in need thereof, one or more agents increasing or inhibiting the expression or activity of one or more MM biomarkers in amounts sufficient to inhibit the progression of multiple myeloma in the subject, wherein the one or more MM biomarkers correspond to one or more gene products identified in expression module 10, expression module 13, expression module 15, and/or expression module 20.

### BRIEF DESCRIPTION OF THE DRAWINGS

FIG. 1 shows bioinformatics pipeline design for the analysis of gene expression and clinical data.

FIG. 2 shows clustering dendrogram of all expressed genes based on consensus topological overlap with the corresponding module colors and associated clinical traits. The figure shows the gene dendrogram obtained by clustering the dissimilarity based on consensus topological overlap with the corresponding module shades indicated by the shade rows. Each shade row (module shades) represents a color-coded module containing a group of highly connected genes. Four biologically significant modules were identified from the 21 modules output from WGCNA. The relationship between each relevant clinical trait was assessed for each color-coded module. Bypassing the default Pearson's correlation method in WGCNA, biweight midcorrelation was applied as a robust alternative implemented in its WGCNA function (bicor).

FIG. 3 shows module-relatedness clustering and module-trait correlations reveal modules associated with multiple myeloma clinical traits. The figure shows the dendrogram of consensus module eigengenes obtained by WGCNA on the consensus correlation and shows the heatmap of module-trait relationships. Each row in the heatmap corresponds to a specific clinical trait and each column to a module. The module shades are shown at the bottom of each column. The boxes shaded darkest are intended to highlight module-trait correlations with a significant p-value of <0.05. Boxes shaded lighter and darker denote negative and positive correlations to MM's vital status, respectively. While the boxes with no shade depict no correlation. A module-trait bicor color scale was used to indicates modules with a significant Student's t-test p-value that cluster together.

FIG. 4 shows box plots of differential expression patterns of module eigengenes across MM patient samples. Each color of the box represents the module and the associated p-values. Royalblue, salmon, and purple modules are positively correlated with vital status of MM patients on standard RVD therapy. However, the midnight blue module is negatively correlated with MM vital status.

FIG. 5 shows volcano plots of differential gene expression identifies gene transcripts that contain upregulated genes, pane A, downregulated genes, Panel B, and both upregulated and downregulated genes, panel C, based on MM vital status for biologically significant modules. The number of downregulated genes transcripts is denoted on the left and the number of upregulated gene transcripts is denoted on the right. The total number of upregulated genes is 676, while the total number of downregulated genes is 121. The log 2 fold change is plotted on the X-axis, and the negative log 10μ-value is plotted on the y-axis. Gene transcripts are colored by module membership.

FIGS. 6A-6B shows string was used to detect predictive interactions between the top fold change genes from the royal blue module. K-means clustering algorithm was used to cluster the genes based on the biological functions. Genes denoted with aqua blue (C1) nodes are mainly from the MAGE (Melanoma Antigen Gene) family of genes, i.e., CTAG2, HBE1, MAGEA1, MAGEA3, MAGEA6, MAGEB2, MAGEC2, PAGE1, PAGE5, and SSX1. Genes clustered with red nodes (C2) are mainly from the Gamma-aminobutyric acid receptor genes, which are major inhibitory neurotransmitters in the mammalian brain, i.e., BDKRB1, GABRA3, GABRB2, GABRAG1, GABRAG2, HTR2C, SOKLH1, and TENM1. To change the size and color of each node, we uploaded the clustered genes into Cytoscape.

FIGS. 7A-7B shows overall survival of differentially expressed hub genes with p-values <0.05, kME >0.7 and Log Fold Change (LFC) ≥0.5 from the M20 module (CTAG2, GABRA3, MAGEA1, MAGEA6, and HTR2C) were analyzed using KMplotter. The patient samples were split based on the median expression. The red lines denote samples with high gene expression, and the black lines refers to samples with low expression. Hazard ratios (HR) are reported for low expression of the genes.

FIG. 8A shows Receiver Operating Characteristic (ROC). ROC curve and corresponding AUC for the COX model obtained for the time dependent (Overall Survival (OS)) predictive abilities of BDKRB1 and GABRA3 from M20. The y-axis is the sensitivity Area Under the Curve (AUC), and the x-axis is OS at a specific point in time. The estimated AUC values for the prognostic tools derived from our COX model are 0.7063 for GABRA3 and 0.6917 for BDKRB1.

FIG. 8B shows a combined Cox and Logistic model provide accurate predictive performance (0.7318) in estimation of time-dependent probabilities for multiple myeloma OS while on standard therapies (RVD) for two years. The x-axis is the OS per day, and the y-axis is AUC percentage. The dark line represents the line of predictive accuracy per number of OS days.

FIG. 9 shows Kaplan-Meier survival analysis of biomarker genes. To analyze the prognostic value of BDKRB1 and GABRA3 genes, expression samples of BDKRB1 and GABRA3 genes from the M20 network were visualized. The expression of BDKRB1 and GABRA3 in the MMRF CoMMpass database was grouped into > or =1 FPKM and <1 FPKM; since BDKRB1 and GABRA3 are only expressed in a subset of patients (1 FPKM is approximately 1 mRNA per cell and while this can vary from cell type to cell type it seems like a reasonable approximation to whether a gene is expressed). The KM plot above shows that patients' Mantel-Cox test determined p-values and hazard ratios (HR) with 95% confidence intervals (CI).

### DETAILED DESCRIPTION

### Reference to Electronic Sequence Listing

The application contains a Sequence Listing which has been submitted electronically in .XML format and is hereby incorporated by reference in its entirety. Said .XML copy, created on Oct. 7, 2025, is named “1013-843 DIV.xml” and is 3,595 bytes in size. The sequence listing contained in this .XML file is part of the specification and is hereby incorporated by reference herein in its entirety.

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting, and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

### I. Definitions

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

A “biomarker,” as used herein, refers to a protein or nucleic acid that can be detected and measured in parts of the body, such as plasma cells or bone marrow where the presence or concentration reflects the presence, severity, type or progression of multiple myeloma in a subject. In molecular terms biomarker is the subset of markers that might be detected in a subject using genomics, proteomics technologies or imaging technologies. A biomarker in accordance with the present application refers to a gene product that is upregulated or downregulated in a subject with multiple myeloma.

“Patient” or “subject” as used herein means a mammalian animal, including a human, a veterinary or farm animal, a domestic animal or pet, and animals normally used for clinical research. In one embodiment, the subject of these methods and compositions is a human. In another embodiment, the subject is a female.

The terms “treat,” “treating” or “treatment” as used herein, refers to a method of alleviating or abrogating a disorder and/or its attendant symptoms. The terms “prevent”, “preventing” or “prevention,” as used herein, refer to a method of barring a subject from acquiring a disorder and/or its attendant symptoms. In certain embodiments, the terms “prevent,” “preventing” or “prevention” refer to a method of reducing the risk of acquiring a disorder and/or its attendant symptoms.

The term “inhibits” is a relative term, an agent inhibits a response or condition if the response or condition is quantitatively diminished following administration of the agent, or if it is diminished following administration of the agent, as compared to a reference agent. Similarly, the term “prevents” does not necessarily mean that an agent completely eliminates the response or condition, so long as at least one characteristic of the response or condition is eliminated. Thus, a composition that reduces or prevents an infection or a response, such as a pathological response, can, but does not necessarily completely eliminate such an infection or response, so long as the infection or response is measurably diminished, for example, by at least about 50%, such as by at least about 70%, or about 80%, or even by about 90% of (that is to 10% or less than) the infection or response in the absence of the agent, or in comparison to a reference agent.

The term “increased level” refers to a level that is higher than a normal or control level customarily defined or used in the relevant art. For example, an increased level of immunostaining in a tissue is a level of immunostaining that would be considered higher than the level of immunostaining in a control tissue by a person of ordinary skill in the art.

The term “decreased level” refers to a level that is lower than a normal or control level customarily defined or used in the relevant art. For example, a decreased level of immunostaining in a tissue is a level of immunostaining that would be considered lower than the level of immunostaining in a control tissue by a person of ordinary skill in the art.

The term “biological sample,” as used herein, refers to material of a biological origin, which may be a body fluid or body product such as blood, plasma, bone marrow, urine, saliva, spinal fluid, stool, sweat or breath. Biological sample also includes tissue samples and cell samples.

“Multiple myeloma (MM),” also known as plasma cell myeloma and simply myeloma, is a cancer of plasma cells, a type of white blood cell that normally produces antibodies. Often, no symptoms are noticed initially. As it progresses, bone pain, anemia, kidney dysfunction, and infections may occur. Complications may include amyloidosis. The cause of multiple myeloma is unknown. Risk factors include obesity, radiation exposure, family history, and certain chemicals. Multiple myeloma may develop from monoclonal gammopathy of undetermined significance that progresses to smoldering myeloma. The abnormal plasma cells produce abnormal antibodies, which can cause kidney problems and overly thick blood. The plasma cells can also form a mass in the bone marrow or soft tissue. When one tumor is present, it is called a plasmacytoma; more than one is called multiple myeloma. Multiple myeloma is diagnosed based on blood or urine tests finding abnormal antibodies, bone marrow biopsy finding cancerous plasma cells, and medical imaging finding bone lesions. Another common finding is high blood calcium levels.

The term “gamma-aminobutyric acid (GABA)” is the major inhibitory neurotransmitter in the mammalian brain where it acts on GABA-A receptors, which are ligand-gated chloride channels. Chloride conductance of these channels can be modulated by agents such as benzodiazepines that bind to the GABA-A receptor (also called herein, interchangeably, GABAA receptor and GABA_{A} receptor). At least 16 distinct subunits of GABA-A receptors have been identified, including the alpha 3 subunit, GABRA3. “GABRA3” is a subunit of the GABAA receptors that may associate with other GABA_{A} receptor subunits to form a functional chloride channel that mediates the inhibitory synaptic transmission in the mature central nervous system (CNS). In the case of the GABAA receptor, there are 16 related subunits (α1-6, β1-3, ∞1-3, δ, ε, θ, π) that comprise the “classical” GABA_{A} receptor plus an additional three subunits (ρ1-3) that form the so-called GABAc receptor. The GABA recognition site occurs at the interface of the α and β subunits and when a ∞2 subunit is adjacent to either an α1, α2, α3 or α5 subunit, a benzodiazepine recognition site is formed. As used herein, GABRA3 refers to the α3 subunit of the GABAa receptor, including functional fragments thereof. See, Atack, J R, Development of Subtype-Selective GABAA Receptor Compounds for the Treatment of Anxiety, Sleep Disorders and Epilepsy, J. M. Monti et al. (eds.), GABA and Sleep, DOI 10.1007/978-3-0346-0226-6_2, #Springer Basel AG 2010, which is incorporated herein by reference.

The term “polynucleotide,” when used in singular or plural form, generally refers to any polyribonucleotide or polydeoxribonucleotide, which may comprise unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs, siRNAs, miRNAs, and shRNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The terms “antisense oligonucleotide” and “ASO” refer to a compound comprising or consisting of an oligonucleotide at least a portion of which is complementary to a target nucleic acid to which it is capable of hybridizing, resulting in at least one antisense activity.

An “antisense activity” refers to any detectable and/or measurable change attributable to the hybridization of an antisense oligonucleotide to its target nucleic acid.

As used herein, the term “heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector. As an example, “heterologous” when used in the context of nucleic acid sequences, such as coding sequences and control sequences, may refer to sequences that are not normally joined together, and/or are not normally associated with one another under ordinary circumstances found in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this application. Allelic variation or naturally occurring mutational events do not give rise to heterologous DNA, as used herein.

A “coding sequence” refers to a nucleic acid sequence which “encodes” a particular protein. The nucleic acid sequence in a polynucleotide is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence is usually be located 3′ to the coding sequence.

The term DNA “control sequences” refers collectively to promoter sequences, polyadenylation signals, transcription termination sequences, upstream regulatory domains, origins of replication, internal ribosome entry sites (“IRES”), enhancers, and the like, which collectively provide for the replication, transcription, and translation of a coding sequence in a recipient cell. Not all of these control sequences need always be present so long as the selected coding sequence is capable of being replicated, transcribed and translated in an appropriate host cell.

The term “promoter region” is used herein in its ordinary sense to refer to a DNA regulatory sequence to which RNA polymerase binds, initiating transcription of a downstream (3′ direction) coding sequence. Further, the term should be broadly construed as additionally encompassing other regulatory elements, including enhancer regions, intron splice donors and acceptors, other 5′ untranslated regions and the like. A promoter sequence may be homologous or heterologous to the desired gene sequence. A wide range of promoters are known and available in the art for the present application, including a wide range of viral and mammalian promoters. Cell type selective or tissue specific promoters can be utilized to target or enhance expression of gene sequences in specific cell populations relative to others. Suitable mammalian and viral promoters. A promoter may be constitutively active, conditionally active, or inducible, depending on the cell type.

“Operably linked” refers to an arrangement of elements wherein the components so described are configured so as to perform their usual function. Thus, control sequences operably linked to a coding sequence are capable of effecting the expression of the coding sequence. Operably linking a heterologous sequence to a promoter, results in a chimeric gene. The control sequences need not be contiguous with the coding sequence, so long as they function to direct the expression thereof. Thus, for example, intervening untranslated yet transcribed sequences can be present between a promoter sequence and the coding sequence, and the promoter sequence can still be considered “operably linked” to the coding sequence.

For the purpose of describing the relative position of a nucleotide sequence in a particular nucleic acid molecule throughout the present application, such as when a particular nucleotide sequence is described as being situated “upstream”, “downstream”, “3′”, or “5′” relative to another sequence, these modifiers should be construed as relating sequence portions in the “sense” or “coding” strand of a DNA molecule, as is conventional in the art.

The term “transgene” refers to a polynucleotide that is introduced into a cell and is capable of being transcribed into RNA and optionally, translated and/or expressed under appropriate conditions. The transgene confers a desired property to a cell into which it was introduced, or otherwise leads to a desired therapeutic or diagnostic outcome. In another aspect, it may be transcribed into a molecule that mediates RNA interference, such as an miRNA, an siRNA, an shRNA, or a guide RNA for CRISPR/Cas9 mediated targeting of the mutant allele.

The term “expression” encompasses the processes by which nucleic acids (e.g., DNA) are transcribed to produce RNA, and (where applicable) RNA transcripts are processed and translated into polypeptides.

The term “gene product” (also referred to herein as “gene expression product” or “expression product”) encompasses products resulting from expression of a gene, such as RNA transcribed from a gene and polypeptides arising from translation of such RNA. It will be appreciated that certain gene products may undergo processing or modification, e.g., in a cell. For example, RNA transcripts may be spliced, polyadenylated, etc., prior to mRNA translation, and/or polypeptides may undergo co-translational or post-translational processing such as removal of secretion signal sequences, removal of organelle targeting sequences, or modifications such as phosphorylation, fatty acylation, etc. The term “gene product” encompasses such processed or modified forms. Genomic, mRNA, polypeptide sequences from a variety of species, including human, are known in the art and are available in publicly accessible databases such as those available at the National Center for Biotechnology Information (www.ncbi.nih.gov) or Universal Protein Resource (www.uniprot.org). Databases include, e.g., GenBank, RefSeq, Gene, UniProtKB/SwissProt, UniProtKB/Trembl, and the like. In general, sequences, e.g., mRNA and polypeptide sequences, in the NCBI Reference Sequence database may be used as gene product sequences for a gene of interest. It will be appreciated that multiple alleles of a gene may exist among individuals of the same species. For example, differences in one or more nucleotides (e.g., up to about 1%, 2%, 3-5% of the nucleotides) of the nucleic acids encoding a particular protein may exist among individuals of a given species. Due to the degeneracy of the genetic code, such variations often do not alter the encoded amino acid sequence, although DNA polymorphisms that lead to changes in the sequence of the encoded proteins can exist. Examples of polymorphic variants can be found in, e.g., the Single Nucleotide Polymorphism Database (dbSNP), available at the NCBI website at www.ncbi.nlm.nih.gov/projects/SNP/. (Sherry S T, et al. (2001). “dbSNP: the NCBI database of genetic variation.” Nucleic Acids Res. 29 (1): 308-311; Kitts A, and Sherry S, (2009). Multiple isoforms of certain proteins may exist, e.g., as a result of alternative RNA splicing or editing. In general, where aspects of this disclosure pertain to a gene or gene product, embodiments pertaining to allelic variants or isoforms are encompassed, if applicable, unless indicated otherwise. Certain embodiments may be directed to particular sequence(s), e.g., particular allele(s) or isoform(s).

As used herein, the term “RNA interference” or “RNAi” refers generally to RNA-dependent silencing of gene expression initiated by double stranded RNA (dsRNA) molecules in a cell's cytoplasm. dsRNA molecule reduces or inhibits transcription products of a target nucleic acid sequence, thereby silencing the gene or reducing expression of that gene.

As used herein, the term “double stranded RNA” or “dsRNA” refers to an RNA molecule having a duplex structure and comprising an effector sequence and an effector complement sequence which are of similar length to one another. The effector sequence and the effector complement sequence can be in a single RNA strand or in separate RNA strands. The “effector sequence” (often referred to as a “guide strand”) is substantially complementary to a mutant target sequence targeted by the first active agent. The “effector sequence” can also be referred to as the “antisense sequence.” The “effector complement sequence” will be of sufficient complementary to the effector sequence such that it can anneal to the effector sequence to form a duplex. In this regard, the effector complement sequence will be substantially homologous to a region of target sequence. As will be apparent to the skilled person, the term “effector complement sequence” can also be referred to as the ‘complement of the effector sequence” or the sense sequence.

As used herein, the term “duplex” refers to regions in two complementary or substantially complementary nucleic acids (e.g., RNAs), or in two complementary or substantially complementary regions of a single-stranded nucleic acid (e.g., RNA), that form base pairs with one another, either by Watson-Crick base pairing or any other manner that allows for a stabilized duplex between the nucleotide sequences that are complementary or substantially complementary. It will be understood by the skilled person that within a duplex region, 100% complementarity is not required; substantial complementarity is allowable. Substantial complementarity may include, for example, 79% or greater complementarity. For example, a single mismatch in a duplex region consisting of 19 base pairs (i.e., 18 base pairs and one mismatch) results in 94.7% complementarity, rendering the duplex region substantially complementary. In another example, two mismatches in a duplex region consisting of 19 base pairs (i.e., 17 base pairs and two mismatches) results in 89.5% complementarity, rendering the duplex region substantially complementary. In yet another example, three mismatches in a duplex region consisting of 19 base pairs (i.e., 16 base pairs and three mismatches) results in 84.2% complementarity, rendering the duplex region substantially complementary, and so on.

The dsRNA may be provided as a hairpin or stem loop structure, with a duplex region comprised of an effector sequence and effector complement sequence linked by at least 2 nucleotide sequence which is termed a stem loop. When a dsRNA is provided as a hairpin or stem loop structure it can be referred to as a “hairpin RNA” or “short hairpin RNAi agent” or “shRNA.” Other dsRNA molecules provided in, or which give rise to, a hairpin or stem loop structure include primary miRNA transcripts (pri-miRNA) and precursor microRNA (pre-miRNA). Pre-miRNA shRNAs can be naturally produced from pri-miRNA by the action of the enzymes Drosha and Pasha which recognize and release regions of the primary miRNA transcript which form a stem-loop structure. Alternatively, the pri-miRNA transcript can be engineered to replace the natural stem-loop structure with an artificial/recombinant stem-loop structure. That is, an artificial/recombinant stem-loop structure may be inserted or cloned into a pri-miRNA backbone sequence which lacks its natural stem-loop structure. In the case of stem-loop sequences engineered to be expressed as part of a pri-miRNA molecule, Drosha and Pasha recognize and release the artificial shRNA. dsRNA molecules produced using this approach are known as “shmiRNAs,” “shmiRs” or “microRNA framework shRNAs”.

As used herein, the term “siRNA” is used with reference to short (20-25 nucleotides), double-stranded RNA molecules that are engineered to use the RNAi pathway to degrade a target mRNA to induce sequence-specific post-transcriptional gene silencing of mRNAs. Synthetically produced siRNAs structurally mimic the types of siRNAs normally processed in cells by the enzyme Dicer. When expressed from a viral vector, the viral vector is engineered to transcribe a short double-stranded hairpin-like RNA (shRNA) that is processed into a targeted siRNA inside the cell. Upon delivery into the cytoplasm, argonaute (AGO)2 cleaves the passenger (sense) strand and the guide (antisense) strand of the siRNA is loaded into the RNA-induced silencing complex (RISC). The guide strand then guides the RISC to the target mRNA which is recognized and cleaved. The RISC and guide strand can be recycled and therefore one siRNA molecule can drive the cleavage of multiple mRNA molecules resulting in highly efficient gene silencing.

As used herein, the term “DNA-directed RNAi construct” or “ddRNAi construct” refers to a nucleic acid comprising DNA sequence which, when transcribed produces a shRNA or shmiR molecule (preferably a shmiR) which elicits RNAi. The ddRNAi construct may comprise a nucleic acid which is transcribed as a single RNA that is capable of self-annealing into a hairpin structure with a duplex region linked by a stem loop of at least 2 nucleotides i.e., shRNA or shmiR, or as a single RNA with multiple shRNAs or shmiRs, or as multiple RNA transcripts each capable of folding as a single shRNA or shmiR respectively. The ddRNAi construct may be provided within a larger “DNA construct” comprising one or more additional DNA sequences. For example, the ddRNAi construct may be provided in a DNA construct further comprising a DNA sequence coding for functional wild type allele which has been genetically altered so that its mRNA transcript is not targeted by RNAi in the ddRNAi construct.

As used herein, the term “complementary” with regard to a sequence refers to a complement of the sequence by Watson-Crick base pairing, whereby guanine (G) pairs with cytosine (C), and adenine (A) pairs with either uracil (U) or thymine (T). A sequence may be complementary to the entire length of another sequence, or it may be complementary to a specified portion or length of another sequence. One of skill in the art will recognize that U may be present in RNA, and that T may be present in DNA. Therefore, an A within either of an RNA or DNA sequence may pair with a U in an RNA sequence or T in a DNA sequence.

As used herein, the term “substantially complementary” is used to indicate a sufficient degree of complementarity or precise pairing such that stable and specific binding occurs between nucleic acid sequences e.g., between the effector sequence and the effector complement sequence or between the effector sequence and the target sequence. It is understood that the sequence of a nucleic acid need not be 100% complementary to that of its target or complement. The term encompasses a sequence complementary to another sequence with the exception of an overhang. Depending on the length of the area of complementarity, a sequence may be complementary to another sequence with the exception of 1 mismatch, 2 mismatches, 3 mismatches, 4 mismatches, 5 mismatches, 6 mismatches, 7 mismatches, 8 mismatches, 9 mismatches, 10 mismatches or more.

The term “encoded,” as used in the context of an shRNA or shmiR of the present application, shall be understood to mean a shRNA or shmiR which is capable of being transcribed from a DNA template. Accordingly, a nucleic acid that encodes, or codes for, a shRNA or shmiR of the present application will include a DNA sequence that serves as a template for transcription of the respective shRNA or shmiR.

As used herein, the terms “transfection,” “gene transfer”, “gene delivery” are used interchangeably herein with reference to methods or systems for inserting foreign nucleic acids into host cells. Gene transfer can result in transient expression of non-integrated transferred DNA, extrachromosomal replication, and expression of transferred replicons (e.g., episomes), or integration of transferred genetic material into the genomic DNA of host cells. Various techniques are known to those of ordinary skill in the art to introduce one or more exogenous nucleic acid molecules, into suitable host cells, including chemical, electrical, and viral-mediated transfection procedures.

The term “gene therapy” is used herein with reference to treatment of a disease or disorder by introducing a suitable polynucleotide into cells in vivo or ex vivo.

The term “host cell” as used herein generally refers to a cell (e.g., bacterial cell, yeast cell, insect cell, mammalian cell) which serves as a recipient for exogenously introduced nucleic acids or has been transfected with an exogenous nucleic acid. It should be understood that the progeny of a single parental cell may not necessarily be completely identical in morphology or in genomic or total DNA complement to the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

A nucleotide or amino acid residue in a first nucleic acid or protein “corresponds to” a residue in a second nucleic acid or protein if the two residues perform one or more corresponding functions and/or are located at corresponding positions in the first and second nucleic acids or proteins. Corresponding functions are typically the same, equivalent, or substantially equivalent functions, taking into account differences in the environments of the two nucleic acids or proteins as appropriate. Residues at corresponding positions typically align with each other when the sequences of the two nucleic acids or proteins are aligned to maximize identity (allowing the introduction of gaps) using a sequence alignment algorithm or computer program such as those referred to below (see “Identity”) and/or are located at positions such that when the 3-dimensional structures of the proteins is superimposed the residues overlap or occupy structurally equivalent positions and/or form the same, equivalent, or substantially equivalent intramolecular and/or intermolecular contacts or bonds (e.g., hydrogen bonds). The structures may be experimentally determined, e.g., by X-ray crystallography or NMR or predicted, e.g., using structure prediction or molecular modeling software. An alignment may be over the entire length of one or more of the aligned nucleic acid or polypeptide sequences or over at least one protein domain (or nucleotide sequence encoding a protein domain).

A “vector” is used herein with reference to a recombinant plasmid or virus that includes a heterologous nucleic acid of interest to be delivered into a host cell, either in vitro or in vivo. The nucleic acid of interest may be linked to, e.g., inserted into, the vector using, e.g., restriction and ligation. Vectors include, for example, DNA or RNA plasmids, cosmos, naturally occurring or modified viral genomes or portions thereof, nucleic acids that can be packaged into viral capsids, mini-chromosomes, artificial chromosomes, etc. Plasmid vectors typically include an origin of replication (e.g., for replication in prokaryotic cells). A plasmid may include part or all of a viral genome (e.g., a viral promoter, enhancer, processing or packaging signals, and/or sequences sufficient to give rise to a nucleic acid that can be integrated into the host cell genome and/or to give rise to infectious virus). Viruses or portions thereof that can be used to introduce nucleic acids into cells may be referred to as viral vectors, which are further described below. A vector may contain one or more nucleic acids encoding a marker suitable for identifying and/or selecting cells that have taken up the vector. Markers include, for example, various proteins that increase or decrease either resistance or sensitivity to antibiotics or other agents (e.g., a protein that confers resistance to an antibiotic such as puromycin, hygromycin or blasticidin), enzymes whose activities are detectable by assays known in the art (e.g., β-galactosidase or alkaline phosphatase), and proteins or RNAs that detectably affect the phenotype of cells that express them (e.g., fluorescent proteins). Vectors often include one or more appropriately positioned sites for restriction enzymes, which may be used to facilitate insertion into the vector of a nucleic acid, e.g., a nucleic acid to be expressed.

An “expression vector” is a vector designed to incorporate a desired nucleic acid of interest in operable linkage to regulatory elements (also termed “regulatory sequences”, “expression control elements”, or “expression control sequences”) mediating expression of the nucleic acid of interest as an RNA transcript (e.g., an mRNA that can be translated into protein or a noncoding RNA such as an shRNA or miRNA precursor). Expression vectors include regulatory sequence(s), e.g., expression control sequences, sufficient to direct transcription of an operably linked nucleic acid under at least some conditions; other elements required or helpful for expression may be supplied by, e.g., the host cell or by an in vitro expression system. Such regulatory sequences typically include a promoter and may include enhancer sequences or upstream activator sequences. In some embodiments a vector may include sequences that encode a 5′ untranslated region and/or a 3′ untranslated region, which may comprise a cleavage and/or polyadenylation signal. In general, regulatory elements may be contained in a vector prior to insertion of a nucleic acid whose expression is desired or may be contained in an inserted nucleic acid or may be inserted into a vector following insertion of a nucleic acid whose expression is desired. Expression vectors include non-viral vectors such as plasmid vectors, and viral vectors such as adeno virus vectors, adeno-associated virus (AAV) vectors, lentivirus vectors, herpes virus vectors.

As used herein, a nucleic acid and regulatory element(s) are said to be “operably linked” when they are covalently linked so as to place the expression or transcription of the nucleic acid under the influence or control of the regulatory element(s). For example, a promoter region would be operably linked to a nucleic acid if the promoter region were capable of effecting transcription of that nucleic acid. One of ordinary skill in the art will be aware that the precise nature of the regulatory sequences useful for gene expression may vary between species or cell types, but may in general include, as appropriate, sequences involved with the initiation of transcription, RNA processing, or initiation of translation. The choice and design of an appropriate vector and regulatory element(s) is within the ability and discretion of one of ordinary skill in the art. For example, one of skill in the art will select an appropriate promoter (or other expression control sequences) for expression in a desired species (e.g., a mammalian species) or cell type. A vector may contain a promoter capable of directing expression in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EF1α, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, a promoter that ordinarily directs transcription by a eukaryotic RNA polymerase II (a “pol II promoter”) or a functional variant thereof is used. In some embodiments, a promoter that ordinarily directs transcription by a eukaryotic RNA polymerase I promoter, e.g., a promoter for transcription of ribosomal RNA (other than 5S rRNA) or a functional variant thereof is used. In some embodiments, a promoter that ordinarily directs transcription by a eukaryotic RNA polymerase III (a “pol III promoter”), e.g., (a U6, H1, 7SK or tRNA promoter or a functional variant thereof) may be used. One of ordinary skill in the art will select an appropriate promoter for directing transcription of a sequence of interest. Examples of expression vectors that may be used in mammalian cells include, e.g., the pcDNA vector series, pSV2 vector series, pCMV vector series, pRSV vector series, pEFI vector series, Gateway® vectors, etc. In some embodiments, regulatable (e.g., inducible, or repressible) expression control element(s), e.g., a regulatable promoter, is/are used so that expression can be regulated, e.g., turned on or increased or turned off or decreased. For example, the tetracycline-regulatable gene expression system (Gossen & Bujard, Proc. Natl. Acad. Sci. 89:5547-5551, 1992) or variants thereof (see, e.g., Allen, N, et al. (2000) Mouse Genetics and Transgenics: 259-263; Urlinger, S, et al. (2000). Proc. Natl. Acad. Sci. U.S.A. 97 (14): 7963-8; Zhou, X., et al (2006). Gene Ther. 13 (19): 1382-1390 for examples) can be employed to provide inducible or repressible expression. Other inducible/repressible systems may be used in various embodiments. For example, expression control elements that can be regulated by small molecules such as artificial or naturally occurring hormone receptor ligands (e.g., steroid receptor ligands such as naturally occurring or synthetic estrogen receptor or glucocorticoid receptor ligands), tetracycline or analogs thereof, metal-regulated systems (e.g., metallothionein promoter) may be used in certain embodiments. In some embodiments, tissue-specific or cell type specific regulatory element(s) may be used, e.g., in order to direct expression in one or more selected tissues or cell types. In some embodiments a vector capable of being stably maintained and inherited as an episome in mammalian cells (e.g., an Epstein-Ban virus-based episomal vector) may be used.

As used herein, the term “viral vector” refers to a recombinant polynucleotide vector comprising virally-derived nucleic acids containing sequences facilitating replication and expression of exogenously incorporated transgene sequences operatively linked to suitable control elements and one or more heterologous sequences (i.e., nucleic acid sequence not of viral origin).

The term “recombinant virus” is used herein with reference to a virus that has been genetically altered, e.g., by the addition or insertion of a heterologous nucleic acid construct into a virus particle.

The term “parvovirus” refers to a DNA animal virus that contains a linear, single-stranded DNA genome, which is classified in the Parvoviridae family, and includes autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, mouse minute virus, bovine parvovirus, canine parvovirus, chicken parvovirus, feline, panleukopenia virus, feline parvovirus, goose parvovirus, and B19 virus. Other autonomous parvoviruses are known to those skilled in the art.

The dependovirus genus includes adeno-associated viruses (AAV), including but not limited to, AAV type 1, AAV type 2, AAV type 3, AAV type 4, AAV type 5, AAV type 6, avian AAV, bovine AAV, canine AAV, equine AAV, and ovine AAV. See, e. g., Bernard N. Fields et al., Virology, vol. 2, ch. 69 (3d ed., Lippincott-Raven Publishers).

The term “wild-type AAV” as used herein refers to both wild-type and pseudo-wild-type AAV. “Pseudo-wild-type AAV” are replication-competent AAV virions produced by either homologous or non-homologous recombination between an AAV vector carrying ITRs and an AAV helper vector carrying rep and cap genes. Pseudo-wild-type AAV have nucleic acid sequences that differ from wild-type AAV sequences.

By “AAV virion” is meant a complete virus particle, such as a wild-type (wt) AAV virus particle (comprising a linear, single-stranded AAV nucleic acid genome associated with an AAV capsid protein coat). In this regard, single-stranded AAV nucleic acid molecules of either complementary sense, i.e., “sense” or “antisense” strands, can be packaged into any one AAV virion and both strands are equally infectious.

The terms “AAV vector” and “recombinant AAV vector (rAAV vector)” are used interchangeably herein with reference to a polynucleotide vector comprising one or more heterologous sequences (i.e., nucleic acid sequence not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a “recombinant adeno-associated viral particle (rAAV particle).”

An “AAV vector” may be derived from any adeno-associated virus serotype, including without limitation, AAV-1, AAV-2, AAV-3, AAV-4, AAV-5, AAV-6, AAV-7, AAV-8, AAV-9 etc. AAV vectors can have one or more of the AAV wild-type genes deleted in whole or part, preferably the rep and/or cap genes, but retain functional flanking ITR sequences. Functional ITR sequences are necessary for the rescue, replication, and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion, or substitution of nucleotides, so long as the sequences provide for functional rescue, replication and packaging.

The terms “recombinant AAV virion,” “rAAV virion,” and “rAAV virus particle” is used interchangeably herein with reference to an infectious, replication-defective virus particle composed of viral particle composed of at least one AAV capsid protein and an encapsidated rAAV vector genome comprising a heterologous nucleotide sequence of interest that is flanked on both sides by AAV ITRs. A rAAV virion is produced in a suitable host cell comprising an AAV vector, AAV helper functions, and accessory functions. A host cell containing these components is capable of encoding AAV polypeptides required for packaging the AAV vector (containing a recombinant nucleotide sequence of interest) into infectious recombinant virion particles for subsequent gene delivery.

By “adeno-associated virus inverted terminal repeats” or “AAV ITRs” is meant the art-recognized regions found at each end of the AAV genome which function together in cis as origins of DNA replication and as packaging signals for the viral genome. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, e.g., Kotin, R. M. (1994) Human Gene Therapy 5, 793-801; Bems, K. I. “Parvoviridae and their Replication” In Fundamental Virology, 2d ed., (B. N. Fields and D. M. Knipe, eds.) for the AAV-2 sequence. As used herein, an “AAV ITR” need not have the wild-type nucleotide sequence depicted in the previously cited references, but may be altered, e.g., by the insertion, deletion, or substitution of nucleotides. Additionally, the AAV ITR may be derived from any of several AAV serotypes, including without limitation, AAV-1, AAV-2, AAV-3, AAV-4, AAV-5, AAV-6, AAV-7, AAV-8, AAV-9 etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell.

By “AAV rep coding region” is meant the art-recognized region of the AAV genome which encodes the replication proteins of the virus which are required to replicate the viral genome and to insert the viral genome into a host genome during latent infection. The term also includes functional homologues thereof such as the human herpesvirus 6 (HHV-6) rep gene which is also known to mediate AAV-2 DNA replication (Thomson et al. (1994) Virology 204, 304-311). For a further description of the AAV rep coding region, see, e.g., Muzyczka, N. (1992) Current Topics in Microbiol. and Immunol. 158, 97-129; Kotin, R. M. (1994) Human Gene Therapy 5, 793-801. The rep coding region, as used herein, can be derived from any viral serotype, such as the AAV serotypes described above. The region need not include all of the wild-type genes but may be altered, e.g., by the insertion, deletion or substitution of nucleotides, so long as the rep genes present provide for sufficient integration functions when expressed in a suitable recipient cell.

The term “long forms of Rep” refers to the Rep 78 and Rep 68 gene products of the AAV rep coding region, including functional homologues thereof. The long forms of Rep are normally expressed under the direction of the AAV p5 promoter.

The term “AAV producer cell” refers to a mammalian or insect cell that can support AAV production.

The phrase “short forms of Rep” refers to the Rep 52 and Rep 40 gene products of the AAV rep coding region, including functional homologues thereof. The short forms of Rep are expressed under the direction of the AAV p19 promoter.

By “AAV cap coding region” is meant the art-recognized region of the AAV genome which encodes the coat proteins of the virus which are required for packaging the viral genome. For a further description of the cap coding region, see, e.g., Muzyczka, N. (1992) Current Topics in Microbiol. and Immunol. 158, 97-129; Kotin, R. M. (1994) Human Gene Therapy 5, 793-801. The AAV cap coding region, as used herein, can be derived from any AAV serotype, as described above. The region need not include all of the wild-type cap genes but may be altered, e.g., by the insertion, deletion or substitution of nucleotides, so long as the genes provide for sufficient packaging functions when present in a host cell along with an AAV vector.

“AAV helper functions” refer to AAV-derived coding sequences that can be expressed to provide AAV gene products that, in turn, function in trans for productive AAV replication. Thus, AAV helper functions include the rep and cap regions. The rep expression products have been shown to possess many functions, including, among others: recognition, binding and nicking of the AAV origin of DNA replication; DNA helicase activity; and modulation of transcription from AAV (or other heterologous) promoters. The cap expression products supply necessary packaging functions. AAV helper functions are used herein to complement AAV functions in trans that are missing from AAV vectors.

The term “AAV helper construct” refers to a nucleic acid molecule that includes nucleotide sequences providing AAV functions deleted from an AAV vector which is to be used to produce a transducing vector for delivery of a nucleotide sequence of interest. AAV helper constructs are commonly used to provide transient expression of AAV rep and/or cap genes to complement missing AAV functions that are necessary for lytic AAV replication; however, helper constructs lack AAV ITRs and can neither replicate nor package themselves. AAV helper constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al. (1989) J. Virology 63, 3822-3828; McCarty et al. (1991) J. Virology 65, 2936-2945. A number of other vectors have been described which encode Rep and/or Cap expression products. See, e.g., U.S. Pat. No. 5,139,941.

The term “accessory functions” refers to non-AAV derived viral and/or cellular functions upon which AAV is dependent for its replication. Thus, the term embraces DNAs, RNAs and protein that are required for AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

Adenovirus-derived accessory functions have been widely studied, and a number of adenovirus genes involved in accessory functions have been identified and partially characterized. Specifically, early adenoviral E1A, E1B 55K, E2A, E4, and VA RNA gene regions are thought to participate in the accessory process. Janik et al. (1981) Proc. Natl. Acad. Sci. USA 78, 1925-1929. Herpesvirus-derived accessory functions have been described. See, e.g., Young et al. (1979) Prog. Med. Virol. 25, 113. Vaccinia virus-derived accessory functions have also been described. See, e.g., Carter, B. J. (1990), supra., Schlehofer et al. (1986) Virology 152, 110-117.

The term “encapsidation essential adenoviral gene product” refers to an adenoviral gene product essential for encapsidating an adenoviral genome to produce infectious adenovirus particles. Encapsidation typically include “late phase” adenoviral gene products. Exemplary encapsidation essential adenoviral gene products include capsid protein IX, encapsidation protein IVa2, protein 13.6, encapsidation protein 52K, capsid protein precursor pIIIa, penton base (capsid protein III), core protein precursor pVII, core protein V, core protein precursor pX, capsid protein precursor pVI, hexon (capsid protein II), protease, hexon assembly protein 100K, protein 33K, encapsidation protein 22K, capsid protein precursor pVIII, protein UXP, and fiber (capsid protein IV).

The term “encapsidation non-essential adenoviral gene product” refers to an adenoviral gene product that is dispensable for encapsidating an adenoviral genome to produce infectious adenovirus particles. Encapsidation non-essential adenoviral gene products are typically translated from adenovirus E1, E2, E3 and E4 transcriptional units. Thus, encapsidation non-essential adenoviral gene products may include gene products expressed from the adenoviral E1 transcriptional unit (e.g., E1A, E1B-19K, E1B-55K); gene products the adenoviral E2/E2A transcriptional unit (e.g., Iva2, pol, pTP, DBP); gene products the adenoviral E3 transcriptional unit (e.g., E3 CR1 alpha0, E3 gp19, E3 14.7 K, E3 RID-beta); gene products from the E4 transcriptional unit (e.g., E4 34K, E4 ORF1, E4 ORFB, E4 ORF3, E4 ORF4, E4 ORF6/7); or a combination thereof.

A “functional homolog” or a “functional equivalent” of a given adenoviral nucleotide region includes similar regions derived from a heterologous adenovirus serotype, nucleotide regions derived from another virus or from a cellular source, and recombinantly produced or chemically synthesized polynucleotides which function in a manner similar to the reference nucleotide region to achieve a desired result. Thus, a functional homolog of an adenoviral VA RNA gene region or an adenoviral E2A gene region encompasses derivatives and analogues of such gene regions—including any single or multiple nucleotide base additions, substitutions and/or deletions occurring within the regions, so long as the homologue retains the ability to provide its inherent accessory function to support AAV virion production at levels detectable above background.

### II. Methods of Biomarker Detection, Diagnosis, and Expression Monitoring For Treatment

Ideal biomarker(s) for multiple myeloma should be specific for multiple myeloma, sensitive (early and immediate release), predictive (proportionate to the extent of injury), robust (accurate and inexpensive), non-invasive and bridge pre-clinical results and clinical validation. The present application is predicated on the discovery of diagnostic bio markers for multiple myeloma and their use as a basis for treatment. The compositions described herein are useful in methods of detection, diagnosis, monitoring, prognosis of multiple myeloma, clinical status of multiple myeloma, and identification of subjects with an increased risk of multiple myeloma metastasis. The information gained from these methods can be used for more effective treatment of multiple myeloma.

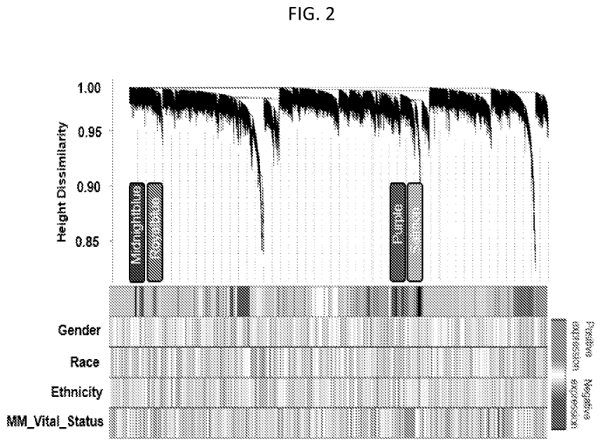

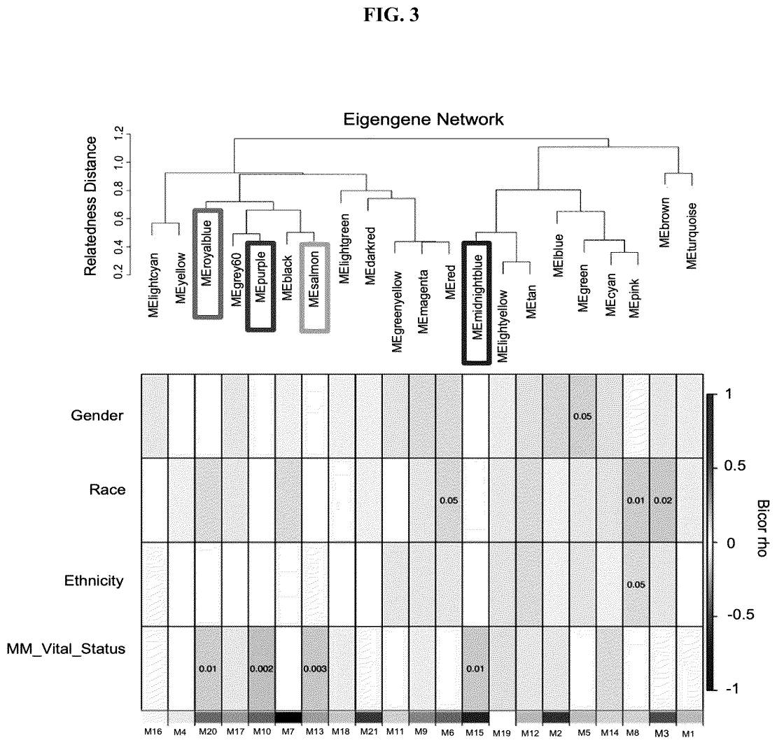

Using MM patient samples provided by Multiple Myeloma Research Foundation (MMRF) CoMMpass dataset, WGCNA and other analysis methods: (i.e. GO_Elite, Kaplan Meier, and Receiver Operating Characteristic curves (ROCs)) were jointly adopted to analyze clinical traits and RNA-Seq data to identify key genes associated with MM vital status. As further described in Example 3, the WGCNA analysis identified twenty-one module eigengenes (MEs), numbered by their rank from the largest number of genes to the smallest, M1 to M21 (FIG. 2). Expression correlation metrics were determined by their relatedness and plotted as a dendrogram (FIG. 3, upper panel). From these 21 modules, upregulation of genes in the M10, M13 and M20 modules and downregulation of genes in the M15 modules was found to be associated with poor survival. The genes in the 4 modules have important clinical implications and serve as diagnostic and prognostic biomarkers or therapeutic targets for treatment of multiple myeloma. In particular, administration of antagonists to M10, M13 and/or M20 module genes and/or administration of agonists to M15 module genes identifies candidate target genes for treatment of multiple myeloma to be used in conjunction with multiple diagnosis treatment.

In one aspect, the present application provides a method for diagnosing multiple myeloma in a subject. The method comprises the steps of (a) measuring the level of one or more biomarkers in a sample from the subject; (b) comparing the level of the one or more biomarkers to a reference level of the one or more biomarkers; (c) making a diagnosis based on the result of the comparing step; and (d) treating the subject with one or more active agents where the subject is diagnosed with multiple myeloma.

In one embodiment, the one or more biomarkers are polynucleotides.

In another embodiment, the one or more biomarkers are proteins or peptides.

In specific embodiments, step (a) includes measuring a panel of 3, 4, 5, 6, 7, 8, 9 or 10 biomarkers in a subject corresponding to one or more modules described herein, namely M10, M13, M15, and/or M20. Upregulation of genes in the M10, M13, and M20 modules and/or downregulation of the M15 module genes is indicative of the presence of multiple myeloma. Active agents for treatment are described below.

Another aspect of the present application relates to a method for determining disease progression or risk for metastasis in a subject with multiple myeloma. In one embodiment, the method comprises the steps of (a) measuring the level of one or more biomarkers in a first sample obtained from the subject with multiple myeloma at a first time point; (b) measuring the level of the one or more biomarkers in a second sample obtained from the subject at a second time point; (c) comparing the level of the one or more biomarkers at the first time point to the level of the one or more biomarkers at the second time point; (d) determining the disease progression between the first and the second time point based on the result of step (c); and (e) further treating the subject with one or more active agents if the multiple myeloma has progressed.

In one embodiment, the one or more biomarkers are polynucleotides.

In another embodiment, the one or more biomarkers are proteins or peptides.

In specific embodiments, step (a) includes measuring a panel of 3, 4, 5, 6, 7, 8, 9 or 10 biomarkers in a subject corresponding to one or more modules described herein, namely M10, M13, M15, and/or M20. Upregulation of genes in the M10, M13, and M20 modules and/or downregulation of the M15 module genes is indicative of the need for further treatment with the active agents described herein. Active agents for treatment are described below.

Another aspect of the present application relates to a method for determining the efficacy of a treatment for multiple myeloma in a subject. The method comprises the steps of (a) measuring the level of one or more biomarkers in a first sample obtained from the subject at a first time point; (b) measuring the level of the one or more biomarkers in a second sample obtained from the subject at a second time point, wherein the subject is under treatment at the second time point; (c) comparing the level of the one or more biomarkers at the first time point to the level of the one or more biomarkers at the second time point; (d) determining the efficacy of the treatment based on the result of step (c); and (e) further treating the subject with one or more active agents if the efficacy has been found to be insufficient for treatment. In certain preferred embodiments, the one or more active agents in step (e) include one or more active agents that were not administered in the previous treatment.

In some embodiments, the one or more biomarkers comprise gene products from module 10. Module 10 genes include NTKR1, MUC1, C1orf226, DCDC1, TGFB2, CRISPLD1, CD109 and NCALD.

In some embodiments, the one or more biomarkers comprise gene products from module 13. Module 13 genes include CBX2, LINC00484, KIF7, TMSB15A, and NEK2.

In some embodiments, the one or more biomarkers comprise gene products from module 15. Module 15 genes include IFITM1, CDH23, AGRN, DHX58, and LINC02576.

In some embodiments, the one or more biomarkers comprise gene products from module 20. Module 20 genes include CTAG2, MAGEA6, GABRB2, SOHLH1, AFAP1-AS1, MAGEA1, CASC9, HTR2C, GLDC and GABRA3.

In another aspect, a method of detecting the risk for multiple myeloma metastasis is provided. The method comprises the steps of (a) measuring the level of one or more biomarkers in a first sample obtained from the subject at a first time point; (b) measuring the level of the one or more biomarkers in a second sample obtained from the subject at a second time point, wherein the subject is under treatment at the second time point; (c) comparing the level of the one or more biomarkers at the first time point to the level of the one or more biomarkers at the second time point; (d) determining the efficacy of the treatment based on the result of step (c); and (e) further treating the subject with one or more active agents if the efficacy has been found to be insufficient for treatment. In certain preferred embodiments, the one or more active agents in step (e) include one or more active agents that were not administered in the previous treatment.

In another embodiment, a method for detecting the risk for multiple myeloma metastasis includes measuring the level of A-to-I RNA edited 3 in a biological sample from the subject, wherein a decrease in the level of A-to-I RNA edited GABRA3 in the sample as compared to a control, is indicative of an increased risk of metastasis. In one embodiment, the control is a sample derived from a subject (or population of subjects) having multiple myeloma. Adenosine deaminases acting on RNA (ADARs) can edit nucleotides in the RNA. Specifically, these enzymes can modify a genetically-encoded adenosine (A) into an inosine (I) in double-stranded RNA structures. ADAR editing results in inosine, which replaces the genomically encoded adenosine, and is read by the cellular machinery as a guanosine (G). Thus, sequencing of inosine-containing RNAs results in G where the corresponding genomic DNA reads A (Bazak et al, doi: 10.1101/gr.164749.113 Genome Res. 2013).

The methods described herein typically employ a patient's sample with a diagnostic reagent, as described above, to form a complex or association with a biomarker in the patients' sample. Detection or measurement of the biomarker may be obtained by use of a variety of apparatuses or machines, such as computer-programmed instruments that can transform the detectable signals generated from the diagnostic reagents complexed with the biomarker binding agent in the biological sample into numerical or graphical data useful in performing the diagnosis or monitoring. Such instruments may be suitably programmed to permit the comparison of the measured biomarker level in the sample with the appropriate reference standard and generate e.g., a diagnostic report or graph.

In certain preferred embodiments, the above methods include the detection of the GABRA3 biomarker in a biological sample from the subject, wherein an increase in the level of GABRA3 in the sample as compared to a control, is indicative of an increased risk of metastasis. In one embodiment, the control is derived from normal plasma. In another embodiment the control is derived from a subject (or population of subjects) with multiple myeloma that has not metastasized.

The presence of the one or more biomarkers in the sample (e.g., GABRA3-ligand complex) may be detected using any assay format known in the art or described herein. There are a variety of assay formats known to the skilled artisan for using a ligand to detect a target molecule in a sample. In general, the upregulation or downregulation of a biomarker in a sample may be determined by (a) contacting the sample with a ligand that interacts with a given biomarker; and (b) determining the level of biomarker expression in the sample, wherein an increase or decrease in a marker (as described above) as compared to a control, is indicative of multiple myeloma, multiple myeloma disease progression, including metastasis, or efficacy of a drug treatment for multiple myeloma. The various assay methods employ one or more binding ligands described herein, e.g., oligo- or polynucleotide probe, PCR amplification product and/or antibody, which detect the biomarker protein or mRNA encoding the same (including fragments or portions thereof).

### Protein Assays

Methods of detection, diagnosis, monitoring, and prognosis of multiple myeloma, or the status of multiple myeloma, and for the identification of subjects with an increased risk of multiple myeloma metastasis by detecting the presence of, or measuring the biomarker level(s) are provided. Such methods may employ polypeptides and/or antibody binding agents as described herein.

The particular assay format used to measure a biomarker protein in a biological sample may be selected from among a wide range of immunoassays, such as enzyme-linked immunoassays, sandwich immunoassays, homogeneous assays, immunohistochemistry formats, or other conventional assay formats. One of skill in the art may readily select from any number of conventional immunoassay formats to perform this assay.

In certain embodiments, the MM biomarkers are detected using enzyme-linked immunosorbent assay (ELISA) which is typically carried out using antibody coated assay plate or wells. Commonly used ELISA assay employs either a sandwich immunoassay or a competitive binding immunoassay.

Briefly, a sandwich immunoassay is a method using two antibodies, which bind to different sites on the antigen or ligand. The primary antibody, which is highly specific for the antigen, is attached to a solid surface. The antigen is then added followed by addition of a second antibody referred to as the detection antibody. The detection antibody binds the antigen to a different epitope than the primary antibody. As a result the antigen is ‘sandwiched’ between the two antibodies. The antibody binding affinity for the antigen is usually the main determinant of immunoassay sensitivity. As the antigen concentration increases the amount of detection antibody increases leading to a higher measured response. The standard curve of a sandwich-binding assay has a positive slope. To quantify the extent of binding different reporters can be used. Typically an enzyme is attached to the secondary antibody which must be generated in a different species than primary antibodies (i.e. if the primary antibody is a rabbit antibody than the secondary antibody would be an anti-rabbit from goat, chicken, etc., but not rabbit). The substrate for the enzyme is added to the reaction that forms a colorimetric readout as the detection signal. The signal generated is proportional to the amount of target antigen present in the sample.

The antibody linked reporter used to measure the binding event determines the detection mode. A spectrophotometric plate reader may be used for colorimetric detection. Several types of reporters have been developed in order to increase sensitivity in an immunoassay. For example, chemiluminescent substrates have been developed which further amplify the signal and can be read on a luminescent plate reader. Also, a fluorescent readout where the enzyme step of the assay is replaced with a fluorophor tagged antibody is becoming quite popular. This readout is then measured using a fluorescent plate reader.

A competitive binding assay is based upon the competition of labeled and unlabeled ligand for a limited number of antibody binding sites. Competitive inhibition assays are often used to measure small analytes. These assays are also used when a matched pair of antibodies to the analyte does not exist. Only one antibody is used in a competitive binding ELISA. This is due to the steric hindrance that occurs if two antibodies would attempt to bind to a very small molecule. A fixed amount of labeled ligand (tracer) and a variable amount of unlabeled ligand are incubated with the antibody. According to law of mass action the amount of labeled ligand is a function of the total concentration of labeled and unlabeled ligand. As the concentration of unlabeled ligand is increased, less labeled ligand can bind to the antibody and the measured response decreases. Thus the lower the signal, the more unlabeled analyte there is in the sample. The standard curve of a competitive binding assay has a negative slope.

In certain other embodiments, the MM biomarkers are detected using antibody coated microbeads. In some embodiments, the microbeads are magnetic beads. In other embodiments, the beads are internally color-coded with fluorescent dyes and the surface of the bead is tagged with an anti-MM biomarker antibody that can bind a MM biomarker in a test sample. The MM biomarker, in turn, is either directly labeled with a fluorescent tag or indirectly labeled with an anti-marker antibody conjugated to a fluorescent tag. Hence, there are two sources of color, one from the bead and the other from the fluorescent tag. Alternatively, the beads can be internally coded by different sizes.

By using a blend of different fluorescent intensities from the two dyes, as well as beads of different sizes, the assay can measure up to hundreds of different MM biomarkers. During the assay, a mixture containing the color/size-coded beads, fluorescence labeled anti-marker antibodies, and the sample are combined and injected into an instrument that uses precision fluidics to align the beads. The beads then pass through a laser and, on the basis of their color or size, either get sorted or measured for color intensity, which is processed into quantitative data for each reaction.

When samples are directly labeled with fluorophores, the system can read and quantitate only fluorescence on beads without removing unbound fluorophores in solution. The assays can be multiplexed by differentiating various colored or sized beads. Real time measurement is achievable when a sample is directly required for unlabeled samples. Standard assay steps include incubation of a sample with anti-marker antibody coated beads, incubation with biotin or fluorophore-labeled secondary antibody, and detection of fluorescence signals. Fluorescent signals can be developed on bead (by adding streptavidin-fluorophore conjugates for biotinylated secondary antibody) and read out by a bead analyzer. Depending on the anti-marker immobilized on the bead surface, a bead-based immunoassay can be a sandwich type or a competitive type immunoassay.

In other embodiments, the MM biomarkers are detected by a protein microarray containing immobilized MM biomarker-specific antibodies on its surface. The microarray can be used in a “sandwich” assay in which the antibody on the microarray captures a MM biomarker in the test sample and the captured marker is detected by a labeled secondary antibody that specifically binds to the captured marker. In a preferred embodiment, the secondary antibody is biotinylated or enzyme-labeled. The detection is achieved by subsequent incubation with a streptavidin-fluorophore conjugate (for fluorescence detection) or an enzyme substrate (for colorimetric detection).

Typically, a microarray assay contains multiple incubation steps, including incubation with the samples and incubation with various reagents (e.g., primary antibodies, secondary antibodies, reporting reagents, etc.). Repeated washes are also needed between the incubation steps. In one embodiment, the microarray assays is performed in a fast assay mode that requires only one or two incubations. It is also conceivable that the formation of a detectable immune complex (e.g., a captured MM biomarker/anti-marker antibody/label complex) may be achieved in a single incubation step by exposing the protein microarray to a mixture of the sample and all the necessary reagents. In one embodiment, the primary and secondary antibodies are the same antibody.

In another embodiment, the protein microarray provides a competitive immunoassay. Briefly, a microarray comprising immobilized anti-marker antibodies is incubated with a test sample in the presence of a labeled MM biomarker standard. The labeled MM biomarker competes with the unlabeled MM biomarker in the test sample for the binding to the immobilized antigen-specific antibody. In such a competitive setting, an increased concentration of the specific MM biomarker in the test sample would lead to a decreased binding of the labeled MM biomarker standard to the immobilized antibody and hence a reduced signal intensity from the label.

The microarray can be processed in manual, semi-automatic or automatic modes. Manual mode refers to manual operations for all assay steps including reagent and sample delivery onto microarrays, sample incubation and microarray washing. Semi-automatic modes refer to manual operation for sample and reagent delivery onto microarray, while incubation and washing steps operate automatically. In an automatic mode, three steps (sample/reagent delivery, incubation and washing) can be controlled by a computer or an integrated breadboard unit with a keypad. For example, the microarray can be processed with a ProteinArray Workstation (PerkinElmer Life Sciences, Boston, Mass.) or Assay 1200™. Workstation (Zyomyx, Hayward, Calif). Scanners by fluorescence, colorimetric and chemiluminescence, can be used to detect microarray signals and capture microarray images. Quantitation of microarray-based assays can also be achieved by other means, such as mass spectrometry and surface plasma resonance. Captured microarray images can be analyzed by stand-alone image analysis software or with image acquisition and analysis software package. For example, quantification of an antigen microarray can be achieved with a fluorescent PMT-based scanner—ScanArray 3000 (General Scanning, Watertown, Mass.) or colorimetric CCD-based scanner—VisionSpot (Allied Biotech, Ijamsville, Md.). Typically, the image analysis would include data acquisition and preparation of assay report with separate software packages. To speed up the whole assay process from capturing an image to generating an assay report, all the analytical steps including image capture, image analysis, and report generation, can be confined in and/or controlled by one software package. Such an unified control system would provide the image analysis and the generation of assay report in a user-friendly manner.

Other reagents for the detection of biomarker proteins in biological samples, include peptide mimetics, synthetic chemical compounds, and quantitative detection of biomarker protein in biological samples by e.g., liquid chromatography (HPLC), immunohistochemistry, etc.

### Nucleic Acid Assays

Methods of detection, diagnosis, monitoring, and prognosis of multiple myeloma, or the status of multiple myeloma, and for the identification of subjects with an increased risk of multiple myeloma metastasis by detecting the presence of, or measuring the level of biomarker mRNA, are provided herein. Such methods include methods based on hybridization analysis of polynucleotides, methods based on sequencing of polynucleotides, proteomics-based methods or immunochemistry techniques. The most commonly used methods known in the art for the detection and quantification of mRNA expression in a sample include PCR-based methods, such as reverse transcription polymerase chain reaction (RT-PCR) or quantitative PCR (qPCR). Other methods include RNAse protection assays, northern blotting, and in situ hybridization.

For example, in some embodiments the PCR-based method may employ a primer or primer-probe set capable of identifying and/or amplifying a GABRA3 nucleic acid sequence or a portion thereof. An example of a primer set capable of identifying and/or amplifying a GABRA3 nucleic acid sequence or a portion thereof is described in Example 1E. Such primers include GABRA3 Forward-5′-GACCACGCCCAACAAGCT-3′ (SEQ ID NO: 1) and Reverse-5″-AGCATGAATTGTTAACCTCATTGTATAGA-3′ (SEQ ID NO: 2). Other suitable primers can be designed by the person of skill in the art and/or obtained commercially based on the GABRA3 nucleic acid sequence. Such sequences are known in the art and can be found, e.g., at NCBI Reference Sequence: NM_000808.3.

Exemplary commercial products for use in the present methods include TRI-REAGENT, Qiagen RNeasy mini-columns, MASTERPURE Complete DNA and RNA Purification Kit (EPICENTRE.RTM., Madison, Wis.), Paraffin Block RNA Isolation Kit (Ambion, Inc.) and RNA Stat-60 (Tel-Test), the MassARRAY-based method (Sequenom, Inc., San Diego, Calif.), differential display, amplified fragment length polymorphism (iAFLP), and BeadArray™ technology (Illumina, San Diego, Calif.) using the commercially available Luminex100 LabMAP system and multiple color-coded microspheres (Luminex Corp., Austin, Tex.) and high coverage expression profiling (HiCEP) analysis.

The selection of the polynucleotide sequences, their length and labels used in the composition are routine determinations made by one of skill in the art in view of the teachings of which genes can form the gene expression profiles suitable for the diagnosis and prognosis of multiple myeloma. For example, useful primer or probe sequences can be at least 13, at least 15, at least 20, at least 30, at least 40 and over at least 50 nucleotides in length. For example, such probes and polynucleotides can be complementary to portions of mRNA sequences encoding the biomarker mRNA. The probes and primers are typically at least 95%, at least 98%, at least 99% or 100% complementary to the mRNA sequences.

In some embodiments, biomarker expression levels are measured using a DNA microarray. Thus, the expression profile biomarkers can be measured in MM samples using microarray technology. Also known as biochip, DNA chip, or gene array, DNA microarray technology allows for identification of gene expression levels in a biologic sample. Typically, cDNAs or oligonucleotides, each representing a given gene, are immobilized on a substrate, e.g., a small chip, bead or nylon membrane, tagged, and serve as probes that will indicate whether they are expressed in biologic samples of interest. The simultaneous expression of thousands of genes can be monitored simultaneously.

In this method, polynucleotide sequences of interest are plated, or arrayed, on a microchip substrate. The arrayed sequences are then hybridized with specific DNA probes from cells or tissues of interest. The source of mRNA can be total RNA isolated from a biological sample. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

The expression profile of biomarkers can be measured in either fresh or paraffin-embedded tumor tissue, or body fluids using microarray technology. In this method, polynucleotide sequences of interest are plated, or arrayed, on a microchip substrate. The arrayed sequences are then hybridized with specific DNA probes from cells or tissues of interest. As with the RT-PCR method, the source of miRNA typically is total RNA isolated from a biological sample. If the source of mRNA is a primary tumor, mRNA can be extracted, for example, from frozen tissue samples, which are routinely prepared and preserved in everyday clinical practice.

In a specific embodiment of the microarray technique, PCR amplified inserts of cDNA clones are applied to a substrate in a dense array. In one aspect, at least 100, 200, 300, 400, 500, 600, 700, 800, 900, 1,000, 1,500, 2,000, 3000, 4000, 5000, 6000, 7000, 8000, 9000, 10,000, 15,000, 20,000, 25,000, 30,000, 35,000, 40,000, 45,000 or at least 50,000 nucleotide sequences are applied to the substrate. Each sequence can correspond to a different gene, or multiple sequences can be arrayed per gene. The microarrayed genes, immobilized on the microchip, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al. (1996) Proc. Natl. Acad. Sci. USA 93(2):106-149). Microarray analysis can be performed by commercially available equipment following manufacturer's protocols, including without limitation the Affymetrix GeneChip technology (Affymetrix, Santa Clara, Calif.), Agilent (Agilent Technologies, Inc., Santa Clara, Calif.), or Illumina (Illumina, Inc., San Diego, Calif.) microarray technology.

The development of microarray methods for large-scale analysis of gene expression makes it possible to search systematically for molecular markers of cancer classification and outcome prediction in a variety of tumor types.

In some embodiments, the Agilent Whole Human Genome Microarray Kit (Agilent Technologies, Inc., Santa Clara, Calif.). The system can analyze more than 41,000 unique human genes and transcripts represented, all with public domain annotations. The system is used according to the manufacturer's instructions.

In some embodiments, the Illumina Whole Genome DASL assay (Illumina Inc., San Diego, Calif.) is used. The system offers a method to simultaneously profile over 24,000 transcripts from minimal RNA input, from both fresh frozen (FF) and formalin-fixed paraffin embedded (FFPE) tissue sources, in a high throughput fashion.

Microarray expression analysis comprises identifying whether a gene or gene product is up-regulated or down-regulated relative to a reference. The identification can be performed using a statistical test to determine statistical significance of any differential expression observed. In some embodiments, statistical significance is determined using a parametric statistical test. The parametric statistical test can comprise, for example, a fractional factorial design, analysis of variance (ANOVA), a t-test, least squares, a Pearson correlation, simple linear regression, nonlinear regression, multiple linear regression, or multiple nonlinear regression. Alternatively, the parametric statistical test can comprise a one-way analysis of variance, two-way analysis of variance, or repeated measures analysis of variance. In other embodiments, statistical significance is determined using a nonparametric statistical test. Examples include, but are not limited to, a Wilcoxon signed-rank test, a Mann-Whitney test, a Kruskal-Wallis test, a Friedman test, a Spearman ranked order correlation coefficient, a Kendall Tau analysis, and a nonparametric regression test. In some embodiments, statistical significance is determined at a p-value of less than about 0.05, 0.01, 0.005, 0.001, 0.0005, or 0.0001. Although the microarray systems used in the methods of the present application may assay thousands of transcripts, data analysis need only be performed on the transcripts of interest, thereby reducing the problem of multiple comparisons inherent in performing multiple statistical tests. The p-values can also be corrected for multiple comparisons, e.g., using a Bonferroni correction, a modification thereof, or other technique known to those in the art, e.g., the Hochberg correction, Holm-Bonferroni correction, idak correction, or Dunnett's correction. The degree of differential expression can also be taken into account. For example, a gene can be considered as differentially expressed when the fold-change in expression compared to control level is at least 1.2, 1.3, 1.4, 1.5, 1.6, 1.7, 1.8, 1.9, 2.0, 2.2, 2.5, 2.7, 3.0, 4, 5, 6, 7, 8, 9 or 10-fold different in the sample versus the control. The differential expression takes into account both overexpression and underexpression. A gene or gene product can be considered up or down-regulated if the differential expression meets a statistical threshold, a fold-change threshold, or both. For example, the criteria for identifying differential expression can comprise both a p-value of 0.001 and fold change of at least 1.5-fold (up or down). One of skill will understand that such statistical and threshold measures can be adapted to determine differential expression by any molecular profiling technique disclosed herein.

Various methods of the present application make use of many types of microarrays that detect the presence and potentially the amount of biological entities in a sample. Arrays typically contain addressable moieties that can detect the presence of the entity in the sample, e.g., via a binding event. Microarrays include without limitation DNA microarrays, such as cDNA microarrays, oligonucleotide microarrays and SNP microarrays, microRNA arrays, protein microarrays, antibody microarrays, tissue microarrays, cellular microarrays (also called transfection microarrays), chemical compound microarrays, and carbohydrate arrays (glycoarrays). DNA arrays typically comprise addressable nucleotide sequences that can bind to sequences present in a sample. MicroRNA arrays, e.g., the MMChips array from the University of Louisville or commercial systems from Agilent, can be used to detect microRNAs. Protein microarrays can be used to identify protein-protein interactions, including without limitation identifying substrates of protein kinases, transcription factor protein-activation, or to identify the targets of biologically active small molecules. Protein arrays may comprise an array of different protein molecules, commonly antibodies, or nucleotide sequences that bind to proteins of interest. Antibody microarrays comprise antibodies spotted onto the protein chip that are used as capture molecules to detect proteins or other biological materials from a sample, e.g., from cell or tissue lysate solutions. For example, antibody arrays can be used to detect biomarkers from bodily fluids, e.g., serum or urine, for diagnostic applications. Tissue microarrays comprise separate tissue cores assembled in array fashion to allow multiplex histological analysis. Cellular microarrays, also called transfection microarrays, comprise various capture agents, such as antibodies, proteins, or lipids, which can interact with cells to facilitate their capture on addressable locations. Chemical compound microarrays comprise arrays of chemical compounds and can be used to detect protein or other biological materials that bind the compounds. Carbohydrate arrays (glycoarrays) comprise arrays of carbohydrates and can detect, e.g., protein that bind sugar moieties. One of skill will appreciate that similar technologies or improvements can be used according to the methods of the present application.

Certain embodiments of the current methods comprise a multi-well reaction vessel, including without limitation, a multi-well plate or a multi-chambered microfluidic device, in which a multiplicity of amplification reactions and, in some embodiments, detection are performed, typically in parallel. In certain embodiments, one or more multiplex reactions for generating amplicons are performed in the same reaction vessel, including without limitation, a multi-well plate, such as a 96-well, a 384-well, a 1536-well plate, and so forth; or a microfluidic device, for example but not limited to, a TaqMan.TM. Low Density Array (Applied Biosystems, Foster City, Calif.). In some embodiments, a massively parallel amplifying step comprises a multi-well reaction vessel, including a plate comprising multiple reaction wells, for example but not limited to, a 24-well plate, a 96-well plate, a 384-well plate, or a 1536-well plate; or a multi-chamber microfluidics device, for example but not limited to a low density array wherein each chamber or well comprises an appropriate primer(s), primer set(s), and/or reporter probe(s), as appropriate. Typically such amplification steps occur in a series of parallel single-plex, two-plex, three-plex, four-plex, five-plex, or six-plex reactions, although higher levels of parallel multiplexing are also within the intended scope of the current teachings. These methods can comprise PCR methodology, such as RT-PCR, in each of the wells or chambers to amplify and/or detect nucleic acid molecules of interest.

Low density arrays can include arrays that detect 10s or 100s of molecules as opposed to 1000s of molecules. These arrays can be more sensitive than high density arrays. In embodiments, a low density array such as a TaqMan.TM. Low Density Array is used to detect one or more gene or gene product in any of Tables 5-12. For example, the low density array can be used to detect at least 1, 2, 3, 4, 5, 6, 7, 8, 9, 10, 15, 20, 25, 30, 40, 50, 60, 70, 80, 90 or 100 genes or gene products selected from any of Tables 5-12.

In some embodiments, the disclosed methods comprise a microfluidics device, “lab on a chip,” or micrototal analytical system (pTAS). In some embodiments, sample preparation is performed using a microfluidics device. In some embodiments, an amplification reaction is performed using a microfluidics device. In some embodiments, a sequencing or PCR reaction is performed using a microfluidic device. In some embodiments, the nucleotide sequence of at least a part of an amplified product is obtained using a microfluidics device. In some embodiments, detecting comprises a microfluidic device, including without limitation, a low density array, such as a TaqMan.TM. Low Density Array. Descriptions of exemplary microfluidic devices can be found in, among other places, Published PCT Application Nos. WO/0185341 and WO 04/011666; Kartalov and Quake, Nucl. Acids Res. 32:2873-79, 2004; and Fiorini and Chiu, Bio Techniques 38:429-46, 2005.

Any appropriate microfluidic device can be used in the methods of the present application. Examples of microfluidic devices that may be used, or adapted for use with molecular profiling, include but are not limited to those described in U.S. Pat. Nos. 7,591,936, 7,581,429, 7,579,136, 7,575,722, 7,568,399, 7,552,741, 7,544,506, 7,541,578, 7,518,726, 7,488,596, 7,485,214, 7,467,928, 7,452,713, 7,452,509, 7,449,096, 7,431,887, 7,422,725, 7,422,669, 7,419,822, 7,419,639, 7,413,709, 7,411,184, 7,402,229, 7,390,463, 7,381,471, 7,357,864, 7,351,592, 7,351,380, 7,338,637, 7,329,391, 7,323,140, 7,261,824, 7,258,837, 7,253,003, 7,238,324, 7,238,255, 7,233,865, 7,229,538, 7,201,881, 7,195,986, 7,189,581, 7,189,580, 7,189,368, 7,141,978, 7,138,062, 7,135,147, 7,125,711, 7,118,910, 7,118,661, 7,640,947, 7,666,361, 7,704,735; U.S. Patent Application Publication 20060035243; and International Patent Publication WO 2010/072410; each of which patents or applications are incorporated herein by reference in their entirety. Another example for use with methods disclosed herein is described in Chen et al., “Microfluidic isolation and transcriptome analysis of serum vesicles,” Lab on a Chip, Dec. 8, 2009 DOI: 10.1039/b916199f.

### Test Sticks

In some other embodiments, the MM biomarkers in a liquid biosample are detected using a test stick or dip stick. The test stick typically contains a fluid impermeable housing and a fluid permeable “stick” having one or more detection zones. In one embodiment, each detection zone contains a dried binding reagent that binds to a MM biomarker in a biosample. In another embodiment, the dried binding reagent is a labeled binding reagent. In another embodiment, the test stick may further comprise a control zone to indicate that the assay test has been carried out satisfactorily, namely the reagents were present in the test stick and that they become mobilized during running the test and have been transported along the flow path. The control zone can also indicate that the reagents within the device are capable of immunochemical interactions, confirming the chemical integrity of the device. This is important when considering the storage and shipment of the device under desiccated conditions within a certain temperature range. The control zone is typically positioned downstream from the detection zone(s) and may, for example, comprise an immobilized binding reagent for a labeled binding reagent. The labeled binding reagent may be present in a mobilizable form upstream from the control zone and detection zone. The labeled binding reagent may be the same or different to the labeled binding reagent for the MM biomarker.

In one embodiment, the test stick comprise a porous sample receiver in fluid connection with and upstream from one or more flow-paths. The porous sample receiver may be common to all assays. Thus a fluid sample applied to the common sample application region of the device is able to travel along the one or more flow-paths to the respective detection zones. The porous sample receiver may be provided within a housing or may at least partially extend out of said housing and may serve for example to collect a body fluid. The porous sample receiver may also act as a fluid reservoir. The porous sample receiving member can be made from any bibulous, porous or fibrous material capable of absorbing liquid rapidly. The porosity of the material can be unidirectional (i.e. with pores or fibers running wholly or predominantly parallel to an axis of the member) or multidirectional (omnidirectional, so that the member has an amorphous sponge-like structure). Porous plastics material, such as polypropylene, polyethylene (preferably of very high molecular weight), polyvinylidene fluoride, ethylene vinylacetate, acrylonitrile and polytetrafluoro-ethylene can be used. Other suitable materials include glass-fiber.

If desired, an absorbent “sink” can be provided at the distal end of the carrier material. The absorbent sink may comprise, for example, Whatman 3MM chromatography paper, and should provide sufficient absorptive capacity to allow any unbound labeled binding reagent to wash out of the detection zone(s). As an alternative to such a sink it can be sufficient to have a length of porous solid phase material which extends beyond the detection zone(s).

Following the application of a binding reagent to a detection zone, the remainder of the porous solid phase material may be treated to block any remaining binding sites. Blocking can be achieved by treatment for example with protein (e.g. bovine serum albumin or milk protein), or with polyvinyl alcohol or ethanolamine, or combinations thereof. To assist the free mobility of the labeled binding reagent when the porous carrier is moistened with the sample, the porous carrier may further comprise a sugar such as sucrose or lactose and/or other substances, such as polyvinyl alcohol (PVA) or polyvinyl pyrrolidone (PVP). Such material may be deposited, for example, as an aqueous solution in the region to which the labeled binding reagent is to be applied. Such materials could be applied to the porous carrier as a first application followed by the application of the label; alternatively, such materials could be mixed with the label and applied to the porous carrier or combinations of both. Such material may be deposited upstream from or at the labeled binding reagent.

Alternatively, the porous carrier may not be blocked at the point of manufacture; instead the means for blocking the porous carrier are included in a material upstream from the porous carrier. On wetting the test strip, the means for blocking the porous carrier are mobilized and the blocking means flow into and through the porous carrier, blocking as the flow progresses. The blocking means include proteins such as BSA and casein as well as polymers such as PVP, PVA as well as sugars and detergents such as Triton-X100. The blocking means could be present in the macroporous carrier material.

The dried binding reagents may be provided on a porous carrier material provided upstream from a porous carrier material comprising the detection zone. The upstream porous carrier material may be macroporous. The macroporous carrier material should be low or non-protein-binding, or should be easily blockable by means of reagents such as BSA or PVA, to minimize non-specific binding and to facilitate free movement of the labeled reagent after the macroporous body has become moistened with the liquid sample. The macroporous carrier material can be pre-treated with a surface active agent or solvent, if necessary, to render it more hydrophilic and to promote rapid uptake of the liquid sample. Suitable materials for a macroporous carrier include plastic materials such as polyethylene and polypropylene, or other materials such as paper or glass-fiber. In the case that the labeled binding reagent is labeled with a detectable particle, the macroporous body may have a pore size at least ten times greater than the maximum particle size of the particle label. Larger pore sizes give better release of the labeled reagent. As an alternative to a macroporous carrier, the labeled binding reagent may be provided on a non-porous substrate provided upstream from the detection zone, said non-porous substrate forming part of the flow-path.

In another embodiment, the test stick may further comprise a sample receiving member for receiving the fluid sample. The sample receiving member may extend from the housing.

The housing may be constructed of a fluid impermeable material. The housing will also desirably exclude ambient light. The housing will be considered to substantially exclude ambient light if less than 10%, preferably less than 5%, and most preferably less than 1%, of the visible light incident upon the exterior of the device penetrates to the interior of the device. A light-impermeable synthetic plastics material such as polycarbonate, ABS, polystyrene, polystyrol, high density polyethylene, or polypropylene containing an appropriate light-blocking pigment is a suitable choice for use in fabrication of the housing. An aperture may be provided on the exterior of the housing which communicates with the assay provided within the interior space within the housing. Alternatively, the aperture may serve to allow a porous sample receiver to extend from the housing to a position external from the housing.

In the foregoing methods, a control level is used as a reference point. The control level can be any of those described herein. In one embodiment, the control level is the level obtained from an individual, or a population of individuals, who are healthy (i.e., who do not have multiple myeloma). In another embodiment, the control level is the level obtained from an individual, or a population of individuals, who have multiple myeloma that has not metastasized.

In any of the foregoing methods, the biological sample may comprise plasma cells or a bone marrow aspiration and bone marrow core biopsy from bone marrow. In other embodiments, the sample is a metastasized tumor sample or a urine sample.

### Kits

Another aspect of the present application relates to a kit for detecting biomarkers for multiple myeloma in a biological sample. The kit comprises (a) reagents for detecting a panel of biomarkers for multiple myeloma. In some embodiments, the panel of biomarkers include a plurality of oligonucleotide probes, polymerase chain reaction (PCR) primers, and/or reagents for PCR reactions, reverse transcriptase PCR (RT-PCR) assays, or quantitative PCR (qPCR). In specific embodiments, the kit includes a panel of 3, 4, 5, 6, 7, 8, 9 or 10 biomarkers in a subject corresponding to one or more modules described herein, namely M10, M13, M15, and/or M20. Alternatively, the panel of biomarkers includes a combination of biomarkers from two, three or all four modules (i.e., M10, M13, M15, and/or M20).

### Determination of Standard Value, Specificity and Sensitivity

In the present application, the standard expression level of an MM biomarker, such as the concentration of an MM biomarker in a biological sample, can be determined statistically. For example, the concentration of an MM biomarker in a biological sample in healthy individuals can be measured to determine the standard concentration of the MM biomarker statistically. When a statistically sufficient population can be gathered, a value in the range of twice or three times the standard deviation (S.D.) from the mean value is often used as the standard value. Therefore, values corresponding to the mean value+2×.S.D. or mean value+3×S.D. may be used as standard values. The standard values set as described theoretically comprise 90% and 99.7% of healthy individuals, respectively.

Alternatively, standard values can also be set based on the actual expression level in the biological sample. Generally, standard values set this way minimize the percentage of false positives, and are selected from a range of values satisfying conditions that can maximize detection sensitivity. Herein, the percentage of false positives refers to a percentage, among healthy individuals, of patients whose concentration of an MM biomarker in a biological sample is judged to be higher or lower than a standard value. On the contrary, the percentage, among healthy individuals, of patients whose concentration of the MM biomarker in a biological sample is judged to be lower or higher, respectively, than a standard value indicates specificity. That is, the sum of the false positive percentage and the specificity is always 1. The detection sensitivity refers to the percentage of patients whose concentration of an MM biomarker in a biological sample is judged to deviate from a standard value, among all MM patients within a population of individuals for whom the presence of MM has been determined.

As used herein, the term “test sensitivity” is the ability of a screening test to identify multiple myeloma and/or its progression, also characterized by being a test with high sensitivity has few false negatives, additionally a test independent of MM prevalence. The test sensitivity is calculated as true positive tests per total affected patients tested, expressed as a percentage. Test specificity” refers to a screening test which is correctly negative in the absence of multiple myeloma, has high specificity and few false positives, is independent of MM prevalence. The test specificity is calculated as true negative tests per unaffected individuals tested, expressed as a percentage.

The term “PPV” (Positive Predictive Value) is the percent of patients with a positive test for multiple myeloma, and thus assesses reliability of positive test. Calculation: 1.PPV=(True positive)/(True+False positives).

The term “NPV” (Negative Predictive Value) refers to patients with a negative test that have not had multiple myeloma, and assesses reliability of negative test. Calculation: 2.NPV=(True negative)/(true and false negatives).

As the relationship shown above indicates, each of the values for sensitivity, specificity, positive predictive value, and negative predictive value, which are indexes for evaluating the diagnostic accuracy, varies depending on the standard value for judging the level of the concentration of an MM biomarker in a biological sample.

A standard value is usually set such that the false positive ratio is low and the sensitivity is high. However, as also apparent from the relationship shown above, there is a trade-off between the false positive ratio and sensitivity. That is, if the standard value is decreased, the detection sensitivity increases. However, since the false positive ratio also increases, it is difficult to satisfy the conditions to have a low false positive ratio. Considering this situation, for example, values that give the following predicted results may be selected as the preferable standard values in the present application: (1) standard values for which the false positive ratio is 50% or less (that is, standard values for which the specificity is not less than 50%) and (2) standard values for which the sensitivity is not less than 20%.

The standard values can be set using receiver operating characteristic (ROC) curve. An ROC curve is a graph that shows the detection sensitivity on the vertical axis and the false positive ratio on the horizontal axis. A ROC curve can be obtained by plotting the changes in the sensitivity and the false positive ratio, which were obtained after continuously varying the standard value for determining the high/low degree of the concentration of an MM biomarker in a biological sample.

The “standard value” for obtaining the ROC curve is a value temporarily used for the statistical analyses. The standard value for obtaining the ROC curve can generally be continuously varied within a range that allows to cover all selectable standard values. For example, the standard value can be varied between the smallest and largest measured MM biomarker values in biological samples from an analyzed population.

Based on the obtained ROC curve, a preferable standard value to be used in the present application can be selected from a range that satisfies the above-mentioned conditions. Alternatively, a standard value can be selected based on a ROC curve produced by varying the standard values from a range that comprises most of the measured MM biomarker level in a biological sample.

### III. Methods of Treatment

### Small Molecule Agonists or Antagonists

In some embodiments, a method for treating multiple myeloma comprises administering to a subject in need thereof one or more small molecule antagonists against one or more gene products identified in the M10, M13 and M20 modules and/or one or more small molecule agonist against gene products identified in the M15 module.

In certain embodiments, the method for treating multiple myeloma comprises administering to a subject in need thereof targets and inhibitors as listed in the tables below, including 1) Gamma-AminoButyric Acid & Neuroactive Ligand Pathway, 2) Neurotrophic Tyrosine Kinase Receptor Pathway, 3) Antibody Dependent Cytotoxicity, CAR T Cell, or CAR NK cell Antigen Targets, and 4) Cell Cycle and Division Drugs Based on CENPF and RRM2 Expression.

TABLE 1A

| Gamma-AminoButyric Acid & | |
|---|---|
| Neuroactive Ligand Pathway | Known inhibitors |
| BDKRB1 is the G protein-coupled receptor | Direct: Icatibant (FDA approved), Safotibant, |
| for bradykinin, which is a 9 aa peptide | and ELN441958 |
| generated during shock, trauma, | Indirect: Perindopril (FDA approved), |
| inflammation, burns, and allergy common in | Captopril (FDA approved), Enalaprilat (FDA |
| chronic pain. | approved), and Ramipril (FDA approved) |
| HTR2C: The 5-HT2C receptor is a subtype | Pizotifen (FDA approved), Mifepristone |
| of 5-HT receptor that binds the endogenous | RU486 (FDA approved), a N- |
| neurotransmitter serotonin (5- | Desmethylclozapine Norclozapine and |
| hydroxytryptamine, 5-HT). This G protein- | clozapine (FDA approved), fluoxetine (FDA |
| coupled receptor (GPCR) is coupled to | approved), citalopram (FDA approved), and |
| Gq/G₁₁ and mediates excitatory | SCH 23390 hydrochloride |
| neurotransmission. HTR2C denotes the | |
| human gene encoding for the receptor, that in | |
| humans is located at the X chromosome. | |
| GABRA3 is a member of the GABA-A | Flumazenil (FDA approved) |
| receptor gene family of heteromeric | |
| pentameric ligand-gated ion channels through | |
| which GABA, the major inhibitory | |
| neurotransmitter in the mammalian brain, | |
| acts. GABA-A receptors are the site of action | |
| of a number of important pharmacologic | |
| agents including barbiturates, | |
| benzodiazepines, and ethanol | |
| GABRB2 and GABRG2 are members of the | Bicuculline |
| GABA-A receptor gene family of heteromeric | |
| pentameric ligand-gated ion channels through | |
| which GABA, the major inhibitory | |
| neurotransmitter in the mammalian brain, | |
| acts. GABA-A receptors are the site of action | |
| of a number of important pharmacologic | |
| agents including barbiturates, | |
| benzodiazepines, and ethanol | |

TABLE 1B

| Neurotrophic Tyrosine Kinase Receptor | |
|---|---|
| Pathway | Known inhibitors/antagonists |
| NTRK1: The neurotrophic tyrosine kinase | Type I inhibitors: Larotrectinib (FDA |
| receptor (NTKR) is a membrane-bound | approved) and entrectinib (FDA approved) |
| receptor that, upon neurotrophin binding, | Type II inhibitors: altiratinib (FDA approved |
| phosphorylates itself and members of the | orphan drug), cabozantinib (FDA approved in |
| MAPK pathway. The presence of this kinase | September 2021) and foretinib. |
| leads to cell differentiation. Mutations in this | |
| gene can affect congenital insensitivity to | |
| pain, anhidrosis, self-mutilating behavior, | |
| cognitive disability and cancer. | |

TABLE 1C
Antibody Dependent Cytotoxicity, CAR T Cell, or CAR NK cell Antigen Targets

PAGE1 and PAGE5 belongs to a PAGE Family Member of genes that are expressed in a
variety of tumors but not in normal tissues, except for some fetal/reproductive tissues, e.g.,
testies.
MAGEB2 is a member of the MAGEB gene family. It is expressed in testis and placenta, and
in a significant fraction of tumors of various histological types. The MAGEB genes are
clustered on chromosome Xp22-p21.
MAGEC2 is a member of the MAGEB gene family. It is not expressed in normal tissues,
except for testis and placenta, and in a significant fraction of tumors of various histological
types.

TABLE 1D

| Cell Cycle and Division Drug Targets | FDA approved inhibitors/antagonists |
|---|---|
| CENPF: Centromeric proteins (CENPs) | Cisplatin, Sunitinib, and Etoposide. |
| family are involved in centromere formation | |
| and kinetochore organization during mitosis | |
| and play an important role in cancers. | |
| RRM2: Ribonucleoside-diphosphate | Ligand inhibitors: clofarabine, fludarabine, |
| reductase subunit M2, also known as | gemcitabine, hudroxyurea. |
| ribonucleotide reductase small subunit, is an | COH29, Didox, Deferasirox, GW8510, DHS |
| enzyme that in humans is encoded by the | Cladribine, gallium nitrate, fludarabine |
| RRM2 gene. High expression of RRM2 is | phosphate, gemcitabine hydrochloride, |
| common in cancers, and RRM2 is identified | tezacitabine, 3-aminopyridine-2- |
| as a tumor promotor and cancer therapeutic | carboxaldehyde thiosemicarbazone (Triapine, |
| target | 3-AP), and GTI2040. |

In certain embodiments, a method for treating multiple myeloma comprises administering to a subject in need thereof one or more small molecule GABAA or GABRA3 receptor antagonists. A number of pharmacological agents exert their effects on GABRAA by binding to recognition sites that are distinct from the endogenous ligand (GABA) binding site. In this regard, the benzodiazepine recognition site is best understood based upon not only the proven clinical efficacy of compounds acting at this site but also the availability of pharmacological tool compounds as well as genetically modified mice. However, other binding sites, including the GABA binding site, the benzodiazepine binding site, the neurosteroid binding site, convulsant binding site, barbiturate binding site, the subunit binding sites, and the ion channel pore are contemplated targets for the GABAA/GABRA3 antagonists discussed herein.

GABAA/GABRA3 antagonists include all agents which either directly or allosterically modulate the inhibitory function of GABAA or GABRA3 receptors. In one embodiment, this includes compounds such as flumazenil, which bind with high affinity but do not affect GABAA/GABRA3-induced chloride currents, exert no physiological effect on GABAA/GABRA3 but can block, or antagonize, the effects of benzodiazepine site agonists or inverse agonists. These compounds are sometimes described as benzodiazepine site antagonists. In another embodiment, the GABAA/GABRA3 antagonists described herein include benzodiazepine site “inverse agonists”, which reduce GABAA/GABRA3-mediated chloride flux.

Many suitable GABAA/GABRA3 antagonists are known in the art. Such antagonists include, without limitation, bicuculline, gabazine, Iso-THAZ, flumazenil, and DMCM. Other antagonists include allopregnanolone, alphaxalone, 3α,5α-THDOC, ganaxolone, org21465, 17PA. Other antagonists include, without limitation, picrotoxinin, picrotin, picrotoxin, TBPS, and PTZ. Other antagonists include pentobarbital, methodhexital, phenobarbital, secobarbital, loreclezole, etomitade, propofol, thiocolchicoside, pentetrazol, and topiramate.

In one embodiment, the GABAA/GABRA3 antagonist is flumazenil. In another embodiment, the GABRA3 antagonist is picrotoxin. In another embodiment, the GABRA3 antagonist is pentetrazol. In another embodiment, the GABAA/GABRA3 antagonist is topiramate.

In some embodiments, the one or more GABAA/GABRA3 antagonists include KRM-II-81 and KRM-II-18B (Lewter et al., ACS Chem. Neurosci. (2017) 8(6):1305-1312); MP-III-024 (Fischer et al., Brain Res Bull, (2017) 131:62-69); Compound 18 (Falk-Petersen et al., Scientific Reports (2020) 10:10078); Compounds 7 and 8 described in Witkin et al., Pharmacol Biochem Behav. (2017) 157:35-40; and oxadiazole 7 and oxazole 9 described in Poe et al., (2016) J. Med. Chem. 59:10800-10806.

### Polynucleotide Agonists or Antagonists

In one embodiment, a method for treating multiple myeloma comprises administering to a subject in need thereof one or more polynucleotide antagonists

As used herein, the term “polynucleotide antagonist” is used with reference to a polynucleotide or oligonucleotide comprising RNA or DNA, which is capable of silencing or reducing the expression of a biomarker mRNA or protein described herein. The term “polynucleotide agonist” is used with reference to a polynucleotide or oligonucleotide comprising RNA or DNA, which is capable of activating the expression of a biomarker mRNA or protein described herein. In some embodiments, the antagonist or agonist comprises a polynucleotide that is overexpressed.

In one embodiment, a method for treating multiple myeloma comprises administering to a subject in need thereof one or more polynucleotide agonists or antagonists for a given biomarker, such as GABRA3. In certain embodiments, the polynucleotide antagonist comprises a single- or double-stranded DNA, a single- or double-stranded RNA, and combinations thereof. The polynucleotide may contain one or more modified bases. In general, the term polynucleotide embraces all chemically, enzymatically and/or metabolically modified forms of unmodified polynucleotides, oligonucleotides, etc. The polynucleotides can be synthesized by chemical methods, prepared by in vitro recombinant DNA-mediated techniques, and by expression of in cells and organisms.

In one aspect, the present application provides an expression vector suitable for expressing a short interfering RNA (siRNA), a short hairpin RNA (shRNA) molecule, an antisense RNA promoting RNAi-mediated gene silencing of GABRA3 or downregulation of GABRA3 expression, a microRNA (miRNA), a ribozyme, a triplex-forming nucleic acid, an external guide sequence (EGS), or an A-to-I RNA edited GABRA3

A biomarker-directed siRNA contains sequences complementary to a corresponding biomarker mRNA sequence, which suppresses gene expression by mRNA degradation. siRNAs are double stranded, typically 21-23 nucleotide small synthetic RNA that mediate sequence-specific gene silencing, i.e., RNA interference (RNAi). The siRNA associates with a multi protein complex called the RNA-induced silencing complex (RISC), during which the “passenger” sense strand is enzymatically cleaved. The antisense “guide” strand contained in the activated RISC then guides the RISC to the corresponding mRNA because of sequence homology and the same nuclease cuts the target mRNA, resulting in specific gene silencing. The design of an siRNA/shRNA preferably avoids seed matches in the 3′UTR of cellular genes to ensure proper strand selection by RISC by engineering the termini with distinct thermodynamic stability. A single siRNA molecule gets reused for the cleavage of many target mRNA molecules.

A conventional siRNA consists of 19-21 nucleotides with two nucleotide overhangs at the 3′ end, usually TT and UU, which are important for recognition by the RNAi machinery. Increasing the length of the dsRNA may enhance its potency, as demonstrated by an in vitro study that dsRNAs with 27 nucleotides were up to 100 times more potent than the conventional siRNAs with 21 nucleotides. The long dsRNAs require processing by Dicer into the shorter siRNAs (hence they are termed as “Dicer-ready” or “Dicer-substrate” siRNAs), which are more efficiently loaded into the RISC, thus facilitating the subsequent gene silencing mechanism. On the other hand, dsRNAs longer than 30 nucleotides can activate the IFN pathway and should be avoided for therapeutic applications.

Small hairpin RNAs (shRNA) are sequences of RNA, typically about 45-80 base pairs in length that include a region of internal hybridization that creates a hairpin structure. shRNA molecules are processed within the cell to form siRNA which in turn knock down gene expression. The benefit of an shRNA is that it can be incorporated into an expression vector and/or integrated into genomic DNA for longer-term or stable expression, and thus longer knockdown of the target mRNA.

In some embodiments, the polynucleotide antagonist is a single-stranded “antisense” oligonucleotide (or ASO) having a sequence complementarity to a biomarker nucleic acid sequence, e.g., a GABRA3 mRNA sequence. The ASO binds or hybridizes to the target sequence and is designed to downregulate or silence mRNA expression. Antisense oligonucleotide technology has been used most often to reduce the amount an mRNA via antisense induced RNase H cleavage or to alter splicing of a pre-mRNA transcript in a cell.

In some embodiments, the antisense oligonucleotide comprises between 15 to 60 nucleotides complementary to the biomarker nucleic acid sense strand. In one embodiment, antisense oligonucleotide comprises between 15 and 30 nucleotides or between 25 and 50 nucleotides complementary to the biomarker nucleic acid sense strand. In other embodiments, the antisense oligonucleotide comprises up to 20 nucleotides, up to 25 nucleotides, up to 30 nucleotides, up to 35 nucleotides, up to 45 nucleotides, or up to 55 nucleotides complementary to the biomarker sequence.

In certain embodiments, the antisense oligonucleotides may comprise or consist of oligonucleotides comprising at least one modified nucleoside. Such modified nucleotides may comprise a modified sugar moiety, a modified nucleobase, or both. In some embodiments, the ASO comprises at least 5, at least 10, at least 15, at least 20, at least 25 or more modified nucleotides relative to the total number of nucleotides in the ASO. In other the ASO comprises less than 25, less than 20, less than 15, less than 10, less than 5, or any range encompassing the foregoing. The modified nucleotides in the ASO may be contiguous or discontiguous. In some embodiments, each of the nucleosides in the ASO is modified. Exemplary modifications and methods for producing the same are disclosed in U.S. Patent Publication No. 2018/0009837, which is incorporated herein by reference in its entirety.

In certain preferred embodiments, the one or more modified nucleotides include a 2′-O-methyl modified sugar moiety and/or a modified internucleoside linkage. In some embodiments, the modified internucleoside linkage is a phosphodiester internucleoside linkage or a phosphorothioate internucleoside linkage.

Numerous methods for optimization of antisense efficiency by finding the most accessible regions of the target molecule exist. Preferably, the antisense molecule binds the target molecule with a dissociation constant (kd) less than or equal to 10-6, 10-8, 10-10, or 10-12. A representative sample of methods and techniques which aid in the design and use of antisense molecules can be found in the following non-limiting list of U.S. Pat. Nos. 5,135,917, 5,994,320, 6,046,319, 6,057,437, and U.S. Patent Publication No. 2018/0009837, all of which are incorporated herein by reference in their entireties.

MicroRNAs (miRNAs) are a class of small noncoding RNAs of ˜21-25 nucleotides in length which are involved in the regulation of gene expression at the posttranscriptional level by degrading their target mRNAs and/or inhibiting their translation. The goal of using synthetic miRNAs (or miRNA mimics) is to achieve the same biological functions as the endogenous miRNAs. Therefore, the synthetic miRNAs should possess the ability to be loaded to RISC and silence the target mRNAs through the natural miRNA signaling pathway. In theory, a single-stranded RNA molecule containing the sequence that is identical to the guide strand of the mature miRNA could be functioned as miRNA mimic. However, the double stranded miRNA containing both guide and passenger strands was found to be 100 to 1,000 times more potent than the single stranded one. The double stranded structure can facilitate the proper loading of the RNA molecule into the RISC, thereby enhancing the gene silencing effect. Therefore, the design of synthetic miRNAs in accordance with the present application relies on the use of miRNA mimics with a duplex structure. Similarly to shRNAs, viral vectors can be used to express the miRNAs of the present application inside the cells.

In one embodiment, the miRNA, miR-92b may be used to downregulate GABRA3 in multiple myeloma cells.

Ribozymes are nucleic acid molecules that can catalyze a chemical reaction, either intramolecularly or intermolecularly. Ribozymes are thus catalytic nucleic acids. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (see, e.g., U.S. Pat. Nos. 5,334,711 and 5,861,288, WO 9858058 and WO 9718312) hairpin ribozymes (see, e.g., U.S. Pat. Nos. 5,631,115 and 6,022,962), and Tetrahymena ribozymes (see, e.g., U.S. Pat. Nos. 5,595,873 and 5,652,107). There are also a number of ribozymes that are not found in natural systems, but which have been engineered to catalyze specific reactions de novo (see, e.g., U.S. Pat. Nos. 5,580,967 and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in U.S. Pat. Nos. 5,646,042, 5,869,253, 5,989,906, and 6,017,756, all of which are incorporated herein by reference in their entireties.

Triplex forming functional nucleic acid molecules are molecules that can interact with either double-stranded or single-stranded nucleic acid. When triplex molecules interact with a target region, a structure called a triplex is formed, in which three strands of DNA form a complex dependent on both Watson-Crick and Hoogsteen base-pairing. Triplex molecules are preferred because they can bind target regions with high affinity and specificity. It is preferred that the triplex forming molecules bind the target molecule with a kd less than 10-6, 10-8, 10-10, or 10-12. Representative examples of how to make and use triplex forming molecules to bind a variety of different target molecules can be found in U.S. Pat. Nos. 5,176,996, 5,683,874, 5,874,566, and 5,962,426, all of which are incorporated herein by reference in their entireties.

External guide sequences (EGSs) are RNA molecules that consist of a sequence complementary to a target mRNA and recruit intracellular ribonuclease P (RNase P), a tRNA processing enzyme, for specific degradation of the target mRNA. EGSs can be designed to specifically target an RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate (see, e.g., WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. USA 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162, all of which are incorporated herein by reference in their entireties.

In another aspect, a method for downregulating GABRA3R expression in multiple myeloma patients includes increasing the level of A-to-I RNA edited GABRA3. Adenosine-to inosine (A-to-I) RNA editing is a modification of double-stranded pre-mRNA catalyzed by adenosine deaminases acting on RNA (ADAR). ADAR enzymes, which include the biologically active ADAR1 and ADAR2, modify a genetically encoded adenosine (A) in double-stranded RNA structures into an inosine (I), read by the cellular machinery as a guanosine (G). A-to-I RNA editing occurs at a multitude of sites on coding as well as non-coding RNA, thereby affecting RNA properties and increasing translational diversity. While most editing sites in primates are in untranslated regions (UTRs), many editing sites in coding regions are conserved across species.

In this regard, adenosine deaminases are known to act on RNA (ADARs) to edit nucleotides in the RNA. Specifically, these enzymes can modify a genetically encoded adenosine (A) into an inosine (I) in double-stranded RNA structures. ADAR editing results in a conversion to inosine, thereby replacing genomically encoded adenosine in GABRA3 so that the nucleotide is read by the cellular machinery as a guanosine (G).

In another aspect, a method for downregulating GABRA3R expression in multiple myeloma patients includes increasing the level of A-to-I RNA edited GABRA3 in a cell. In one embodiment, a nucleic acid sequences encoding A-to-I RNA edited GABRA3 is engineered into an expression vector for expressing the A-to-I RNA edited GABRA3 in MM cells in a MM patient.

Expression and/or Delivery of Polynucleotide Agonists or Antagonists

A suitable expression vector may be used to express and/or deliver the polynucleotide antagonists of the present application to a subject in need of treatment for multiple myeloma. In certain embodiments, an expression vector, such as a viral vector encodes an siRNA/shRNA or miRNA containing an effector sequence of at least 17-80 contiguous nucleotides, which is substantially complementary to a region of the targeted RNA transcript to facilitate silencing.

In some embodiments, the one or more expression vectors may further encode one or more additional active agents providing additive or synergistic effects during treatment. Alternatively, a therapeutic composition comprising the one or more recombinant viruses may be administered in combination with one or more small molecule drugs indicated for treatment of multiple myeloma.

In certain embodiments, the one or more expression vectors are engineered to direct expression of the polynucleotide antagonist ubiquitously (constitutively). Exemplary promotors for ubiquitous expression, include, but are not limited to, the cytomegalovirus (CMV) immediate early promoter, an RSV LTR, a MoMLV LTR, a phosphoglycerate kinase-1 (PGK) promoter, a simian virus 40 (SV40) promoter, a CK6 promoter, a transthyretin promoter (TTR), a TK promoter, a tetracycline responsive promoter (TRE), a U6 promoter, an E2F promoter, a telomerase (hTERT) promoter, an H1 promoter, a cytomegalovirus enhancer/chicken beta-actin/rabbit β-globin promoter (CAG) promoter, an elongation factor 1-alpha promoter (EF1-α) promoter, a human β-glucuronidase promoter, a chicken 0-actin (CBA) promoter, a retroviral Rous sarcoma virus (RSV) LTR promoter, a dihydrofolate reductase promoter, a 13-actin promoter and the like.

In certain embodiments, the one or more expression vectors are engineered to provide tissue-specific expression of the polynucleotide antagonist to myeloma cells, including a variety of B cell promoters known to be active in plasma cells, plasmablasts, lymphoplasmacytoid cells, memory B cells, B-2 cells, Follicular (FO) B cells, Marginal zone (MZ) B cells, B-1 cells, and regulatory B (Breg) cells

In some embodiments, a nucleic acid encoding an active agent may include operably linked natural or heterologous signal peptide domain for secretion of the active agent from cells. The signal peptide sequence is removed from the mature peptide as the mature peptide is secreted from the cell. Since a given signal peptide sequence can affect the level of peptide expression, a peptide-encoded polynucleotide may include any one of a variety of different N-terminal signal peptide sequences known in the art.

In certain embodiments, the one or more expression vectors are non-viral vectors. In some embodiments, the non-viral vectors are plasmids.

In certain embodiments, the one or more expression vectors are viral vectors. Viral vectors for expression of the polynucleotide antagonist may be derived from, e.g., adenoviruses, adeno-associated viruses (AAV), retroviruses (including lentiviruses, such as HIV-1 and HIV-2), vaccinia viruses and other poxviruses, herpesviruses (e.g., herpes simplex virus Types 1 and 2), polioviruses, Sindbis and other RNA viruses, alphaviruses, astroviruses, coronaviruses, orthomyxoviruses, papovaviruses, paramyxoviruses, parvoviruses, picornaviruses, togaviruses and others. The viral vectors may or may not contain sufficient viral genetic information and/or structural components for production of infectious virus when introduced into host cells, i.e., viral vectors may be replication-competent or replication-defective. In some embodiments, e.g., where structural components for production of infectious virus are lacking, the necessary functional components may be supplied in trans by a host cell or by another vector introduced into the cell when production of recombinant virus is desired. In preferred embodiments, replication-defective recombinant viruses are administered for treatment. A nucleic acid for delivery may be incorporated into a naturally occurring or modified viral genome (or a portion thereof) or may be present within a viral capsid as a separate nucleic acid molecule.

In certain cases, the viral vectors may be engineered to target specific cells, such as plasma cells or multiple myeloma cells by using the targeting characteristics inherent to the virus vector or engineered into the virus vector. Specific cells may be “targeted” for delivery and expression of polynucleotides. Thus, “targeting,” in this case, relates to the use of endogenous or heterologous binding agents in the form of capsids, envelope proteins, antibodies for delivery to specific cells, the use of tissue-specific regulatory elements for restricting expression to specific subset(s) of cells, or both.

In some embodiments, the viral vectors are AAV vectors. AAV vectors provide a preferred delivery system for the nucleic acid therapeutics of the present application since they can allow for long lasting and continuous expression of functional alleles and silencing of the corresponding mutant alleles. AAV vectors can include or can be modified to control expression of the first and second active agents under a number of different regulatory elements, including various promoter and/or enhancer elements for constitutive or cell-type specific expression.

In certain preferred embodiments, the viral vector is a recombinant adenovirus-associated virus (AAV). AAVs are small (20-26 nm) replication-defective, nonenveloped viruses, which depend on the presence of a second virus, such as adenovirus or herpes virus or suitable helper functions, for replication in cells. AAVs are not known to cause disease and induces a very mild immune response. AAVs can infect both dividing and non-dividing cells and may incorporate its genome into that of the host cell. More than 30 naturally occurring serotypes of AAV are available. Many natural variants in the AAV capsid exist, allowing identification and use of AAV vectors with properties specifically suited for the cell targets of delivery. AAV vectors are relatively non-toxic, provide efficient gene transfer, and can be easily optimized for specific purposes. AAV viruses may be engineered using conventional molecular biology techniques to optimize the generation of recombinant AAV particles for cell specific delivery of the transgenes of the present application, for minimizing immunogenicity, enhancing stability, delivery to the nucleus, etc.

Any suitable AAV serotype or AAV pseudotypes may be used to express the polynucleotide antagonists of the present application in cells in vitro and in vivo. The types of vectors for in vivo delivery are preferably chosen based on lack of pre-existing immunity in the host to a selected AAV subtype and stable expression in vivo. Typically, AAV vectors are derived from single-stranded (ss) DNA parvoviruses that are nonpathogenic for mammals. Among the serotypes of AAVs isolated from human or non-human primates, human serotype 2 is the first and best characterized AAV that was developed as a gene transfer vector. Other useful AAV serotypes include AAV1, AAV3, AAV4, AAV5, AAV6, AAV7, AAV8, AAV9, AAV9.47, AAV9(hul4), AAV10, AAV11, AAV12, AAVrh8, AAVrh10, AAV-DJ8 and AAV-DJ.

In some embodiments, the AAV vector may be a pseudotyped AAV vector containing sequences and/or components originating from at least two different AAV serotypes. Thus, a pseudotyped (or chimeric) AAV vector may include, for example, an AAV2-derived genome in an AAV1-derived or AAV6-derived capsid; or an AAV2-derived genome in an AAV4-derived capsid; or an AAV2-derived genome in an AAV9-derived capsid. Alternatively, a pseudotyped AAV vector may include a portion of the capsid from one AAV serotype fused to a second portion of a different AAV serotype capsid, resulting in a vector encoding a pseudotyped AAV2/AAV5 capsid. In other embodiments, a pseudotyped AAV vector may include a capsid from one serotype and inverted terminal repeats (ITRs) from another AAV serotype. Exemplary AAV vectors include recombinant pseudotyped AAV2/1, AAV2/2, AAV2/5, AAV2/7, AAV2/8, and AAV2/9 serotype vectors.

Generally, recombinant AAV-based vectors are replication defective, because they have the rep and cap (capsid) viral genes removed (which account for 96% of the viral genome), leaving the two flanking 145-basepair (bp) inverted terminal repeats (ITRs), which are used to initiate viral DNA replication, packaging and integration. Typically, an AAV vector can accommodate a “minigene” of about 4.5 kb in length comprising one or more transgenes, each operably linked to one or more regulatory elements for expression.

Unless otherwise specified, the AAV ITRs and other selected AAV components described herein, may be readily selected from among any of the aforementioned serotypes or other known or as yet unknown AAV serotypes. These ITRs or other AAV components may be readily isolated from an AAV serotype using standard techniques known to those of ordinary skill in the art. In addition, AAV sequences may be isolated or obtained from academic, commercial, or public sources (e.g., the American Type Culture Collection, Manassas, Va.) or may be obtained through synthetic or other suitable means by reference to published sequences such as are available in the literature or in databases such as, e.g., GenBank, PubMed and the like.

Since ITRs of an AAV vector recombine during the replication process, a revertant phenotype may result in which both ITRs regain wild type sequences. To alleviate this problem, ITRs of different AAV vectors can be used, such as e.g., an AAV2 left ITR with an AAV4 deleted right ITR, etc. The sole criterion governing the choice of ITRs to be combined lies in the sequence identity between the ITRs of the serotype. The ITRs of serotypes 2 and 5 are nearly identical, and the ITRs of serotypes 2 and 4 have an 81.6% similarity. After deletion of the D sequence and trs, the sequence identity between the ITRs of AAV 2 and AAV 4 drops to just over 50%. The combination of these two ITRs therefore generates sufficiently divergent ITRs and will result in an scAAV vector that can no longer regenerate progeny with wildtype ITRs.

Exemplary AAV fragments for assembly into vectors and/or helper cells include the capsid subunit proteins, vp1, vp2, vp3, hypervariable regions, and the rep proteins, rep 78, rep 68, rep 52, and rep 40. When providing the AAV rep and cap products, the AAV rep and AAV cap sequences can both be of one serotype origin, e.g., all AAV8 origin or may be derived from multiple AAV serotypes. In one embodiment, the rep and cap sequences are expressed from separate sources (e.g., separate vectors, or a host cell and a vector). In some embodiments, these rep sequences may be fused in frame to cap sequences of a different AAV serotype to form a chimeric AAV vector, such as AAV2/8 described in U.S. Pat. No. 7,282,199.

A recombinant adeno-associated virus (rAAV) may be generated by culturing a host cell which contains a nucleic acid sequence encoding an adeno-associated virus (AAV) serotype capsid protein, or fragment thereof, as defined herein; a functional rep gene; a vector composed of, at a minimum, AAV inverted terminal repeats (ITRs) and the fusion protein encoding nucleic acid sequence; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. For example, a stable host cell may be generated which is derived from 293 cells (which contain E1 helper functions under the control of a constitutive promoter), but which contains the rep and/or cap proteins under the control of constitutive or inducible promoters.

The minigene, rep sequences, cap sequences, and helper functions required for producing a recombinant AAV (rAAV) of the present disclosure may be delivered or contained in a packaging host cell or helper cell. The selected genetic element may be delivered using any suitable method, including those described herein and any others available in the art. Non-limiting methods of generating rAAV virions are well known in the art.

The polynucleotide antagonists of the present application may be encoded on the same or different recombinant viruses. In some embodiments, a plurality of recombinant viruses may be employed, each encoding e.g., one or more siRNA/shRNA/miRNA/antisense RNA expression units, each targeting a different portion of a biomarker, such as GABRA3.

In some embodiments, the viral titer of the pharmaceutical composition is at least about any of 5×10¹², 6×10¹², 7×10¹², 8×10¹², 9×10¹², 10×10¹², 11×10¹², 15×10¹², 20×10¹², 25×10¹², 30×10¹², or 50×10¹² genome copies/mL.

In some embodiments, the viral titer of the pharmaceutical composition is encompassed in a range between 5×10¹² to 6×10¹², 6×10¹² to 7×10¹², 7×10¹² to 8×10¹², 8×10¹² to 9×10¹², 9×10¹² to 10×10¹², 10×10¹² to 11×10¹², 11×10¹² to 15×10¹², 15×10¹² to 20×10¹², 20×10¹² to 25×10¹², 25×10¹² to 30×10¹², 30×10¹² to 50×10¹², or 50×10¹² to 100×10¹² genome copies/mL. In some embodiments, the viral titer of the pharmaceutical composition is encompassed in a range from about 5×10¹² to 10×10¹², 10×10¹² to 25×10¹², or 25×10¹² to 50×10¹² genome copies/mL.

In some embodiments, the viral titer of the pharmaceutical composition for administration is at least about 5×10⁹, 6×10⁹, 7×10⁹, 8×10⁹, 9×10⁹, 10×10⁹, 11×10⁹, 15×10⁹, 20×10⁹, 25×10⁹, 30×10⁹, or 50×10⁹ transducing units/mL. In some embodiments, the viral titer of the pharmaceutical composition for administration is encompassed by a range from about 5×10⁹ to 6×10⁹, 6×10⁹ to 7×10⁹, 7×10⁹ to 8×10⁹, 8×10⁹ to 9×10⁹, 9×10⁹ to 10×10⁹, 10×10⁹ to 11×10⁹, 11×10⁹ to 15×10⁹, 15×10⁹ to 20×10⁹, 20×10⁹ to 25×10⁹, 25×10⁹ to 30×10⁹, 30×10⁹ to 50×10⁹, or 50×10⁹ to 100×10⁹ transducing units/mL.

In some embodiments, the viral titer of the pharmaceutical composition for administration is at least about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰, 30×10¹⁰, 40×10¹⁰, or 50×10¹⁰ infectious units/mL. In some embodiments, the viral titer of the pharmaceutical composition for administration is encompassed by a range from about 5×10¹⁰ to 6×10¹⁰, 6×10¹⁰ to 7×10¹⁰, 7×10¹⁰ to 8×10¹⁰, 8×10¹⁰ to 9×10¹⁰, 9×10¹⁰ to 10×10¹⁰, 10×10¹⁰ to 11×10¹⁰, 11×10¹⁰ to 15×10¹⁰, 15×10¹⁰ to 20×10¹⁰, 20×10¹⁰ to 25×10¹⁰, 25×10¹⁰ to 30×10¹⁰, 30×10¹⁰ to 40×10¹⁰, 40×10¹⁰ to 50×10¹⁰, or 50×10¹⁰ to 100×10¹⁰ infectious units/mL. In some embodiments, the viral titer of the pharmaceutical composition for administration is encompassed by a range from about 5×10¹⁰ to 10×10¹⁰, 10×10¹⁰ to 15×10¹⁰, 15×10¹⁰ to 25×10¹⁰, or 25×10¹⁰ to 50×10¹⁰ infectious units/mL

In yet another embodiment, the agent is a functional nucleic acid. Functional nucleic acids are nucleic acid molecules that have a specific function, such as binding a target molecule or catalyzing a specific reaction. The functional nucleic acid molecules can act as inhibitors of a specific activity possessed by a target molecule. Functional nucleic acid molecules can interact with any macromolecule, such as DNA, RNA, and polypeptides. Thus, functional nucleic acids can interact with mRNA or the genomic DNA of a given biomarker to inhibit expression or interact with the biomarker protein to inhibit activity. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule but is based on the formation of tertiary structure that allows specific recognition to take place. Examples of functional nucleic acid molecules include siRNA, antisense molecules, aptamers, ribozymes, triplex forming molecules, and external guide sequences.

### Administration

The antagonist may be administered to the subject with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. In certain embodiments, the antagonist is administered directly to a tumor or cancer tissue, including administration directly to the tumor bed during invasive procedures. The antagonist may also be placed on a solid support such as a sponge or gauze for administration against the target chemokine to the affected tissues.

Antagonists can be administered in the usually accepted pharmaceutically acceptable carriers. Acceptable carriers include, but are not limited to, saline, buffered saline, glucose in saline. Solid supports, liposomes, nanoparticles, microparticles, nanospheres or microspheres may also be used as carriers for administration of the antagonists.

One or more of the biomarker agonists or antagonists discussed herein may be administered in combination with other pharmaceutical agents, as well as in combination with each other. The term “pharmaceutical” agent as used herein refers to a chemical compound which results in a pharmacological effect in a patient. A “pharmaceutical” agent can include any biological agent, chemical agent, or applied technology which results in a pharmacological effect in the subject.

The therapeutic compositions administered by these methods are administered directly into the environment of the targeted cell undergoing unwanted proliferation, e.g., a cancer cell or targeted cell (tumor) microenvironment of the patient. Conventional and pharmaceutically acceptable routes of administration include, but are not limited to, systemic routes, such as intraperitoneal, intravenous, intranasal, intravenous, intramuscular, intratracheal, subcutaneous, and other parenteral routes of administration or intratumoral or intranodal administration. Routes of administration may be combined, if desired. In some embodiments, the administration is repeated periodically.

The therapeutic agents of the present application, i.e., antagonists or other selected antagonists, may be administered to a patient, preferably suspended in a biologically compatible solution or pharmaceutically acceptable delivery vehicle. The various components of the compositions are prepared for administration by being suspended or dissolved in a pharmaceutically or physiologically acceptable carrier such as isotonic saline; isotonic salts solution or other formulations that will be apparent to those skilled in such administration. The appropriate carrier will be evident to those skilled in the art and will depend in large part upon the route of administration. Other aqueous and non-aqueous isotonic sterile injection solutions and aqueous and non-aqueous sterile suspensions known to be pharmaceutically acceptable carriers and well known to those of skill in the art may be employed for this purpose.

Because the compositions do not have to cross the blood-brain-barrier, alternate compositions can be provided which do not meet the characteristics required to do so, yet still inhibit the action of a given biomarker target. Thus, in yet another aspect, a method of screening molecules for use in cancer therapy comprises contacting a mammalian cancer or tumor cell culture which expresses a biomarker of the present application, such as GABRA3 or other selected targets with a potential therapeutic molecule, e.g., a small molecule, peptide, polynucleotide, antibody, or the like; and culturing the cell. The culture is then tested for inhibition of cellular migration. Cellular migration assays are known to one of skill in the art. Other methods are known in the art. If cellular migration is decreased as compared to a control, the molecule has an anti-tumor or anti-cancer effect or prevents or reduces cancer metastasis. The level of cellular migration in the test cell culture can be compared to the level of cellular migration in untreated cancer/tumor cell cultures.

### Dosage

The appropriate dosage (“therapeutically effective amount”) of the agonist or antagonist will depend, for example, on the condition to be treated, the severity and course of the condition, whether the antagonist is administered for preventive or therapeutic purposes, previous therapy, the patient's clinical history and response to the agonist or antagonist, the type of agonist or antagonist used, and the discretion of the attending physician. The antagonist is suitably administered to the patent at one time or over a series of treatments and may be administered to the patent at any time from diagnosis onwards. The agonist or antagonist may be administered as the sole treatment or in conjunction with other drugs or therapies useful in treating the condition in question.

As a general proposition, a therapeutically effective amount of the biomarker agonist(s) or antagonist(s) will be administered individually or collectively in the range of about 1 ng/kg body weight/day to about 100 mg/kg body weight/day whether by one or more administrations. In a particular embodiments, the range of antibody administered is from about 1 ng/kg body weight/day to about 1 g/kg body weight/day, 1 ng/kg body weight/day to about 100 ng/kg body weight/day, 1 ng/kg body weight/day to about 10 ng/kg body weight/day, 10 ng/kg body weight/day to about 1 μg/kg body weight/day, 10 ng/kg body weight/day to about 100 ng/kg body weight/day, 100 ng/kg body weight/day to about 1 μg/kg body weight/day, 100 ng/kg body weight/day to about 10 μg/kg body weight/day, 1 g/kg body weight/day to about 10 μg/kg body weight/day, 1 μg/kg body weight/day to about 100 μg/kg body weight/day, 10 μg/kg body weight/day to about 100 μg/kg body weight/day, 10 μg/kg body weight/day to about 1 mg/kg body weight/day, 100 μg/kg body weight/day to about 10 mg/kg body weight/day, 1 mg/kg body weight/day to about 100 mg/kg body weight/day and 10 mg/kg body weight/day to about 100 mg/kg body weight/day.

In another embodiment, the biomarker agonist(s) or antagonist(s) are administered individually or collectively at a dosage range of 1 ng-10 ng per injection, 10 ng to 100 ng per injection, 100 ng to 1 g per injection, 1 g to 10 g per injection, 10 g to 100 g per injection, 100 g to 1 mg per injection, 1 mg to 10 mg per injection, 10 mg to 100 mg per injection, and 100 mg to 1000 mg per injection. The antagonist may be injected daily, or every 2, 3, 4, 5, 6 and 7 days, or every 1, 2, 3 or 4 weeks.

In another particular embodiment, the dose range of the biomarker agonist(s) or antagonist(s) may range from about 1 ng/kg to about 100 mg/kg In still another particular embodiment, the range of antagonist, such as an antibody administered is from about 1 ng/kg to about 10 ng/kg, about 10 ng/kg to about 100 ng/kg, about 100 ng/kg to about 1 μg/kg, about 1 μg/kg to about 10 μg/kg, about g/kg to about 100 μg/kg, about 100 μg/kg to about 1 mg/kg, about 1 mg/kg to about 10 mg/kg, about 10 mg/kg to about 100 mg/kg, about 0.5 mg/kg to about 30 mg/kg, and about 1 mg/kg to about 15 mg/kg.

In other particular embodiments, the biomarker agonist(s) or antagonist(s) is administered individually or collectively in an amount of about, 0.0006, 0.001, 0.003, 0.006, 0.01, 0.03, 0.06, 0.1, 0.3, 0.6, 1, 3, 6, 10, 30, 60, 100, 300, 600 and 1000 mg/day. As expected, the dosage will be dependent on the condition, size, age, and condition of the patient.

The biomarker agonist(s) or antagonist(s) may be administered, as appropriate or indicated, a single dose as a bolus or by continuous infusion, or as multiple doses by bolus or by continuous infusion. Multiple doses may be administered, for example, multiple times per day, once daily, every 2, 3, 4, 5, 6 or 7 days, weekly, every 2, 3, 4, 5 or 6 weeks or monthly. However, other dosage regimens may be useful. The progress of this therapy is easily monitored by conventional techniques.

The dosages and treatment regimens utilizing the biomarker agonist(s) or antagonist(s) of the present application can be determined by the person of skill in the art. Certain of the GABRA3 antagonists are approved for use for the treatment of other conditions, and thus dosages and prescribing information is known. For example, in the case of flumazenil, in one embodiment, a dosage of from about 10 nM to about 10 M is provided to treat multiple myeloma. In another embodiment, a dosage of 0.4 mg-1.0 mg IV is provided.

The dosage required for the biomarker agonist(s) or antagonist(s) depends primarily on factors such as the condition being treated, the age, weight, and health of the patient, and may thus vary among patients. The effective dosage of each active component is generally individually determined, although the dosages of each compound can be the same. In one embodiment, the small molecule dosage is about 1 g to about 1000 mg. In one embodiment, the effective amount is about 0.1 to about 50 mg/kg of body weight including any intervening amount. In another embodiment, the effective amount is about 0.5 to about 40 mg/kg. In a further embodiment, the effective amount is about 0.7 to about 30 mg/kg. In still another embodiment, the effective amount is about 1 to about 20 mg/kg. In yet a further embodiment, the effective amount is about 0.001 mg/kg to 1000 mg/kg body weight. In another embodiment, the effective amount is less than about 5 μg/kg, about 500 mg/kg, about 400 mg/kg, about 300 mg/kg, about 200 mg/kg, about 100 mg/kg, about 50 mg/kg, about 25 mg/kg, about 10 mg/kg, about 1 mg/kg, about 0.5 mg/kg, about 0.25 mg/kg, about 0.1 mg/kg, about 100 μg/kg, about 75 μg/kg, about 50 μg/kg, about 25 μg/kg, about 10 ag/kg, or about 1 ag/kg. However, the effective amount of the biomarker agonist(s) or antagonist(s), as well as dosages different than that used for e.g., brain-related conditions, can be determined by the attending physician, and depends on the condition treated, the compound administered, the route of delivery, age, weight, severity of the patient's symptoms and response pattern of the patient.

Toxicity and therapeutic efficacy of the compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays (such as those described in the examples below) and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the present application, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Formulations

As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions. In certain embodiments, the pharmaceutically acceptable carrier comprises serum albumin.

The pharmaceutical composition of the application is formulated to be compatible with its intended route of administration. Examples of routes of administration include parenteral, e.g., intrathecal, intra-arterial, intravenous, intradermal, subcutaneous, oral, transdermal (topical) and transmucosal administration. In certain embodiments, the pharmaceutical composition is administered directly into a tumor tissue.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active, ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially, for example, from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the application is dictated by and directly dependent on the unique characteristics of the active compound and the particular therapeutic effect to be achieved, and the limitations inherent in the art of compounding such an active compound for the treatment of individuals.

The present application is further illustrated by the following examples that should not be construed as limiting. The contents of all references, patents, and published patent applications cited throughout this application, as well as the Figures and Tables, are incorporated herein by reference.

### Examples

### Materials And Methods

### Data Curation and Normalization

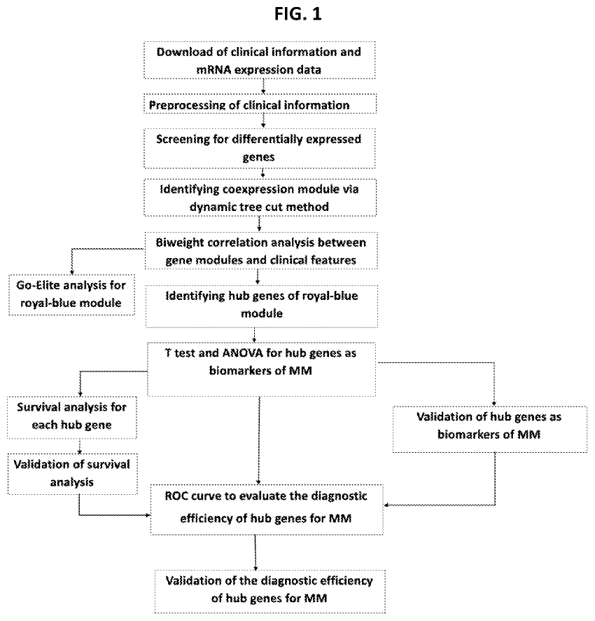

Level 3 deidentified, RNA-seq fragments per kilobase per million mapped reads (FPKM) data was obtained from The Cancer Genome Atlas (TCGA) MMRF CoMMpass study, in which bone marrow samples were collected from newly diagnosed MM patients with informed consent and IRB approval (Barwick, B. G., et al., Cell of Origin and Genetic Alterations in the Pathogenesis of Multiple Myeloma. Front Immunol, 2019. 10: p. 1121). An overview of the clinical trait details for the newly diagnosed MM patients, who were initially treated with RVD combination therapy (Tables 1A-C) and expression data availability (n=270), is provided in (Table 1D). The RNA-seq and clinical data were downloaded on or before Sep. 27, 2019 and analyzed using a bioinformatics pipeline (FIG. 1).

TABLE 2A

| Treatment Name | Number of Patients |
|---|---|
| Bortezomib | 31 |
| Bortezomib-Carfilzomib-Lenenalidomide- | 4 |
| Cyclophosphomide-Dexamethasone | |
| Bortezomib-Carfilzomib-Lenenalidomide- | 1 |
| Dexamethasone | |
| Bordezomib-Cyclophosphomide- | 136 |
| Dexamethasone | |
| Bortezomib-Dexamethasone | 72 |
| Bortezomib-Lenalidomide | 4 |
| Bortezomib-Lenalidomide- | 35 |
| Cyclophosphomide-Dexamethasone | |
| Bortezomib-Lenalidomide- | 289 |
| Dexathmetasone | |
| Bortezomib-Lenalidomide-Melphalan- | 0 |
| Dexamethasone | |
| Bortezomib-Melphalan | 2 |
| Bortezomib-Melphalan-Prednisolone | 28 |
| Bortezomib-Thalidomide-Dexamethasone | 14 |
| Carfilzomib-Cyclophosphomide- | 42 |
| Dexamethasone | |
| Carfilzomib-Dexamethasone | 20 |
| Carfilzomib-Lenalidomide | 7 |
| Carfilzomib-Lenalidomide- | 80 |
| Cyclophosphomide-Dexamethasone | |
| Carfilzomib-Lenalidomide- | 103 |
| Dexamethasone | |
| Cyclophosphomide-Dexamethasone | 18 |
| Lenalidomide | 81 |
| Lenalidomide-Clardribine- | 9 |
| Dexamethasone | |
| Lenalidomide-Dexamethasone | 59 |

TABLE 2B

| Responses | Number Patient VS Best Response |
|---|---|
| Complete Response | 145 |
| Partial Response | 119 |
| Progressive Disease | 8 |
| Stable Disease | 38 |
| Stringent Complete Response | 43 |
| Very Good Partial Response | 368 |
| N/A | 47 |

TABLE 2C

| Responses | Number Patient VS False Response |
|---|---|
| Complete Response | 9 |
| Partial Response | 376 |
| Progressive Disease | 0 |
| Stable Disease | 0 |
| Stringent Complete Response | 6 |
| Very Good Partial Response | 284 |
| N/A | 93 |

TABLE 2D
Distribution of reported clinical traits among MM patients
who received RVD therapy (some died within 2 years).
The number of the patients is 270 (n = 270) after
the data is normalized and the outliers were removed.

| Clinical Traits | Classification | Number of Patients |
|---|---|---|
| Gender | Male | 156/270 |
| | Female | 114/270 |
| Race | European American | 184/270 |
| | African American | 34/270 |
| | Others | 52/270 |
| Tumor stages | I | 83/270 |
| | II | 87/270 |
| | III | 93/270 |
| | IV | 7/270 |
| MM vital status | Alive | 212/270 |
| | Dead | 58/270 |

### Detecting Low Counts, Batch Effect Correction, and Removal of Outliers

The sequencing, alignment, transcript counting, and FPKM for each patient was performed using RNA-seq data as previously described (Griffen, T. L., et al., Multivariate transcriptome analysis identifies networks and key drivers of chronic lymphocytic leukemia relapse risk and patient survival. BMC Med Genomics, 2021. 14(1): p. 171); Ohandjo, A. Q., et al., Transcriptome Network Analysis Identifies CXCL13-CXCR5 Signaling Modules in the Prostate Tumor Immune Microenvironment. Sci Rep, 2019. 9(1): p. 14963]. MMRF CoMMpass MM RNA-seq FPKM data from multiple research centers (baseline case samples) with 60,478 gene-wise short read-based quantifications were curated to address potential technical or site-based variance. Batch effect control, normalization, and quality assessment tests were performed using a Tunable Approach for Median Polish of Ratio (TAMPOR): https://github.com/edammer/TAMPOR [Dill, C. D., et al., A network approach reveals driver genes associated with survival of patients with triple-negative multiple myeloma. iScience, 2021. 24(5): p. 102451; Johnson, E. C. B., et al., Large-scale proteomic analysis of Alzheimer's disease brain and cerebrospinal fluid reveals early changes in energy metabolism associated with microglia and astrocyte activation. Nat Med, 2020. 26(5): p. 769-780.]. All samples were processed together in the sample-gene transcript matrix to capture MM biological variance and preserve it through normalization. TAMPOR maintained the integrity of the data through robust batch effect correction by removing batch artifacts manifesting as batch-wise variance, genes with ≥50% missing or zero values. After this, samples with ≥50% zero values, technical replicates, and cluster outliers (Klimienė, I., et al., Adhesion molecule immunophenotype of bone marrow multiple myeloma plasma cells impacts the presence of malignant circulating plasma cells in peripheral blood. Int J Lab Hematol, 2021. 43(3): p. 403-408) were removed.

### Coding Clinical Metadata for Biological Network Analysis

The clinical traits dataset contains phenotypes for each sample. The non-numeric variables were converted into numeric values. For instance, Gender (“1”=“Male”, “2”=“Female”, “N.A.”=“Unknown”), Race (“1”=“European American”, “2”=“African American”, “3=“Others”), Ethnicity (“1”=“Hispanic or Latino”, “2”=“Not Hispanic or not Latino”, “3”=“Others”), and MM vital status (“0”=“Alive”, “1”=“Dead”).

### Gene Clustering and Network Analysis

WGCNA is an R package used to identify gene co-expression networks in the MMRF CoMMpass study by robustly calculating the eigengene, bicor rho, and p-values for each module and then correlating the first principal component of each module (module eigengenes) with clinical traits or phenotypes of interest. The WGCNA package (WGCNA_1.70-3) was installed from the Comprehensive R Archive Network (CRAN), and all analyses were carried out in R version 3.6, with some system calls to Python v2.7. To reduce RNA-seq data dimensionality from thousands of genes (60,478 to 30,598 genes, n=270 for RVD therapy-receiving patients) to a few modules, WGCNA was used to assess gene co-expression profiles across all MM samples.

A sample dissimilarity matrix (1-topology overlap) was constructed by WGCNA and genes that have similar expression patterns were grouped within the sample cohort. The network was constructed using the WGCNA blockwiseModules function [Langfelder, P., and S. Horvath, WGCNA: an R package for weighted correlation network analysis. BMC Bioinformatics, 2008. 9: p. 559.], with parameters as follows: WGCNA dynamic tree-cutting algorithm, CutreeHybrid, power=7, deepsplit=2, minModuleSize=180, mergeCutHeight=0.15, TOMDenom=“mean”, corType=“bicor”, networkType=“signed”, pamStage=TRUE, pamRespectsDendro=TRUE, reassignThresh=0.05, verbose=3, saveTOMs=FALSE, maxBlockSize larger than the number of genes being clustered (30,598), and reassignThresh=0.05. To limit the impact of high technical variation within RNA-seq data representing differences in transcript abundances across samples, biweight midcorrelation (bicor) was used instead of Pearson correlation to provide robust correlations with less weight given to outliers (Langfelder, P., and S. Horvath, WGCNA: an R package for weighted correlation network analysis. BMC Bioinformatics, 2008. 9: p. 559). The WGCNA R-script and outputs for this study can be downloaded from: https://github.com/pog240/MMRF-WGCNA-ANALYSIS/.

### Gene Ontology (GO) Enrichment and Upstream Regulator Analysis

Gene Ontology Elite (GO Elite) (version 1.2.5) was used to perform gene set enrichment analysis on the biologically significant modules (M10, M13, M15, and M20) to identify overall module enrichment of biological functions, molecular processes, and cellular locations http://www.genmapp.org/go_elite/help_main.htm. GO enrichment analysis used the Ensembl database (Version 62) of pre-defined gene lists organized by biological process, molecular function, and cellular component. Fisher's exact test, adjusted for false discovery, was used to determine overrepresentation or significant overlap between WGCNA modules of interest members and pre-defined gene lists. The reference background list was the subset of 30,598 genes with symbols in the final cleaned-up abundance matrix (22,456 symbols). Additionally, gene set enrichment analysis (GSEA) was performed using the GSEA molecular signature C2 database (version 6.2) to identify associations between network modules and curated lists of genes related to various diseases, particularly cancer. The GSEA C3 database was also used to identify upstream regulators among the genes of interest in each module (Zambon, A. C., et al., GO-Elite: a flexible solution for pathway and ontology over-representation. Bioinformatics, 2012. 28(16): p. 2209-10).

### Differential Gene Expression Analysis

Differential expression via two independent sample t-tests, with equal variance, was conducted to identify gene candidates within significant modules (upregulated and downregulated modules) correlated with MM vital status (poor outcomes). An independent t-test was used to compare differential gene expression among patients who died (n=58) versus those who survived within two years (n=212) on RVD treatments. Data for bone marrow samples from patients who missed treatment or lacked vital status information were excluded from differential expression analysis. False discovery rate (FDR) adjustment was performed using the Benjamini-Hochberg method, with threshold set at FDR <0.01 for the comparison. 22,515 genes were visualized using the EnhancedVolcano R package and used for further analysis.

Protein-Protein Interaction within Modules of Interest

To determine known and predicted Protein-Protein Interaction (PPI) of significant DEGs in our modules of interest (M10, M13, M15, and M20), a Protein-Protein Interaction functional clustering enrichment analysis was performed using genes with a log fold change of 1.9 and above. This analysis was performed using the String database (version 11.0 https://string-db.org/). Genes clustered based on their biological function (k-means) were further analyzed using Enrichr, a web tool, to visualize their collective functions. This PPI enhanced insight into the biological functions of genes in the modules of interest (Szklarczyk, D., et al., The STRING database in 2017: quality-controlled protein-protein association networks, made broadly accessible. Nucleic Acids Res, 2017. 45(D1): p. D362-d368).

### Geneset Enrichment Analysis

Enrichr, a comprehensive gene set enrichment server: https://maayanlab.cloud/Enrichr/was used to visualize clustergrams and to understand the overall biological knowledge for further biological discovery on gene symbol list within modules of interest [Kuleshov, M. V., et al., Enrichr: a comprehensive gene set enrichment analysis web server 2016 update. Nucleic Acids Res, 2016. 44(W1): p. W90-7].

### ROC Analyses

A Receiver Operator Curve (ROC) analysis was performed using the top over-expressed genes in modules positively correlated with MM vital status to determine if the differentially expressed genes could serve as predictive indicators of MM vital status (M10, M13, and M20). EasyROC web tool on the default non-parametric test setting was used to perform ROC.

### Survival Analyses

The prognostic value of MM patients treated with RVD for two years (n=270) was evaluated using KM plotter (www.kmplot.com). MM expression data and their survival information for the samples were uploaded onto the KM plotter web-based tool. To analyze OS of MM patients, patient's samples were split into two groups by median expression (high versus low expression) and assessed by a Kaplan-Meier survival plot, with the hazard ratio (HR) with 95% confidence intervals (CIs) and a log-rank (Mantel-Cox) test was used to determine p-values for both sets of KM analyses.

### Example 1: Transcriptomic Analysis Defines a Network of MM Co-Expression Modules

The transcriptome comprising 30,598 genes across 270 MM case samples was examined for co-expression modules of gene transcripts and the network biology of MM poor outcomes was assessed. The distribution of reported clinical traits among MM patients and their therapy distribution was recorded in Tables 2A-C. These clinical traits were obtained from the MMRF CoMMpass dataset. WGCNA identified twenty-one module eigengenes (MEs), numbered by their rank from the largest number of genes to the smallest, M1 to M21 (FIG. 2; Table 3), expression correlation metric was determined by their relatedness and plotted as a dendrogram (FIG. 3, upper panel). The relatedness dendrogram shows that M16 is closely related to M4. M20 is closely related to M17, MiG, M7, and M13. M18 and M21 are separated but are closely related to M11, M9, and M6. Out of these modules, MiG, M13 and M20 are positively associated with poor survival, while M15 is negatively associated with poor survival.

TABLE 3
Table of Modules

| Module Name | Module Number | Number of Cluster Gene per Module |
|---|---|---|
| turquoise | M1 | 1861 |
| blue | M2 | 1295 |
| brown | M3 | 1274 |
| yellow | M4 | 1246 |
| green | M5 | 1216 |
| red | M6 | 1037 |
| black | M7 | 938 |
| pink | M8 | 931 |
| magenta | M9 | 840 |
| purple | M10 | 690 |
| greenyellow | M11 | 659 |
| tan | M12 | 635 |
| salmon | M13 | 568 |
| cyan | M14 | 560 |
| midnightblue | M15 | 534 |
| lightcyan | M16 | 531 |
| grey60 | M17 | 528 |
| lightgreen | M18 | 494 |
| lightyellow | M19 | 435 |
| royalblue | M20 | 307 |
| darkred | M21 | 216 |

### Example 2: Identification of Transcript Significant Modules Associated with Mortality and Functional Annotation

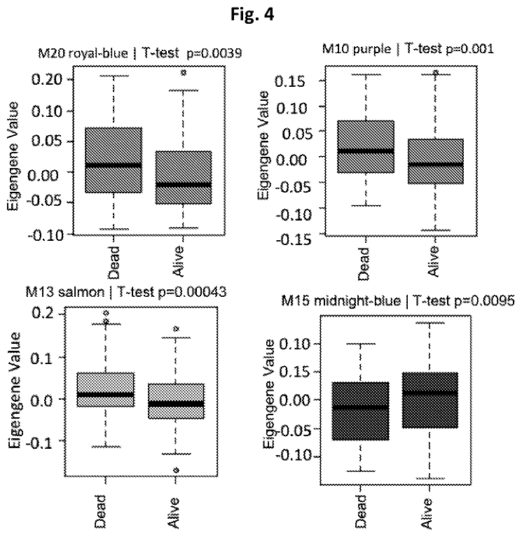

The association of Module Eigengenes (MEs) to MM vital status was assessed by correlation. This helps determine which modules are candidates for molecular causality of the trait of interest in MM patients treated with RVD for two years (Table 2). 21 modules and corresponding module eigengenes were constructed to determine the module networks of interest associated with vital status. The robust correlation of the 21 modules to vital status helped determine the transcriptome networks of interest. The darker shade (red in original) represents positive gene expression, while the lighter shade (blue in original) represents negative gene expression (FIG. 2 heatmap). These analyses identified positive correlations with vital status in the M20 (r=0.150, p=0.0039) with 307 genes, the M13 (r=0.18, p=0.00043) with 568 genes, and the M10 (r=0.18, p=0.001) with 690 genes, but a negative correlation with vital status for the M15 (r=−0.15, p=0.0095) with 534 genes. Additionally, a t-test was performed to compare the expression of MM patients who died and those alive for two years on RVD treatments (FIG. 4).

The top biological functions associated with the full list of M20 members are related to MM progression, and they include skeletal system morphogenesis, G protein-coupled receptor (GPCR) protein signaling pathways, multicellular organismal development, synaptic transmission, and cell-cell adhesion. GPCR kinase 6 (GRK6) was found to be associated with MM progression and implicated as a kinase required for the survival of MM cells. The top biological processes and 10 most significant genes from modules M20, M13, M10, and M15 were listed in Table 4.

TABLE 4

| | | | | Top 20 Up and Down |
|---|---|---|---|---|
| | | Top Biological | GO-Elite | Regulated Genes from T-test |
| | Module | Process | FET FDR | Results |
| Positively correlated | Royal-blue | Skeletal system | 4.10E−4 | Up: CTAG2; MAGEA6; |
| with multiple | P = 0.0030 | morphogenesis | 3.20E−4 | GABRB2; SOHLH1; AFAP1- |
| myeloma vital status | Rho = 0.15 | G-protein coupled | 7.00E−4 | AS1; MAGEA1; |
| | | receptor protein | 4.00E−3 | CASC9; HTR2C; GLDC; |
| | | Multicellular | 6.50E−3 | GABRA3; MAGEA3; |
| | | organismal | | SLCO1A2; BCHE; |
| | | development | | MYO18B; SEMA3A; |
| | | Synaptic transmission | | STK32A; SEMA3D; CD3E; |
| | | Cell-cell adhesion | | PAGE1; BDKRB1 |
| | | | | Down: NONE |
| | Purple | Sterol biosynthetic | 1.10E−3 | Up: NTRK1; CCND2; NES; |
| | P = 0.001 | process | 2.33E−3 | PKP2; C1orf226; DCDC1; |
| | Rho = 0.18 | GPI anchor metabolic | 1.30E−3 | TGFB2; |
| | | process | 1.45E−3 | CRISPLD1; CD109; NCALD; |
| | | Cellular membrane | 2.73E−3 | MUC1; NOS1AP; PLAAT2; |
| | | fusion | | TSPAN12; |
| | | Regulation of cellular | | WSCD1; EVC; LGALS14; |
| | | amino acid metabolic | | INSRR; MDH1B; TSPEAR |
| | | process | | DOWN: NONE |
| | | C-terminal protein | | |
| | | amino acid | | |
| | | modification | | |
| | Salmon | cell cycle phase | 8.00E−113 | Up: CBX2; LINC00484; KIF7; |
| | P = 4.3E−4 | cell cycle | 4.08E−112 | TMSB15A; NEK2; RRM2; |
| | Rho = 0.18 | DNA strand | 2.80E−34 | CENPF; RNU6- |
| | | elongation | 8.63E−46 | 583P; KIF14; FAM72C; E2F8; |
| | | DNA replication | 1.24E−50 | NUF2; KIF20A; RNA5SP323; |
| | | cell division | | IQGAP3; |
| | | | | KIF4A; DRP2; TLCD3B; |
| | | | | E2F2; FAM72D |
| | | | | DOWN: |
| Negatively correlated | Midnight-blue | response to virus | 3.26E−4 | Up: NONE |
| with multiple | P = 0.0095 | Regulation of B cell | 4.26E−5 | DOWN: XXYLT1-AS2; |
| myeloma vital status | Rho = −0.15 | activation | 1.73E−4 | LINC00996; KCNMB2; |
| | | regulation of | 2.94E−4 | MIR320D1; CHST3; FLT3; |
| | | lymphocyte | 1.69E−3 | MTMR9LP; ZC3H12D; |
| | | differentiation | | MIR4420; TRIM22; NUS1P2; |
| | | Hemopoiesis | | MIR339; STAP1; |
| | | cellular | | SYCP2L; IFI44L; IFIT3; |
| | | macromolecule | | CCND1; SAMD7; NCOA4P2; |
| | | catabolic process | | MYO5C |

### Example 3: Modules Differentiating Between Alive and Dead Patients Represent Known Biological Groundworks of MM

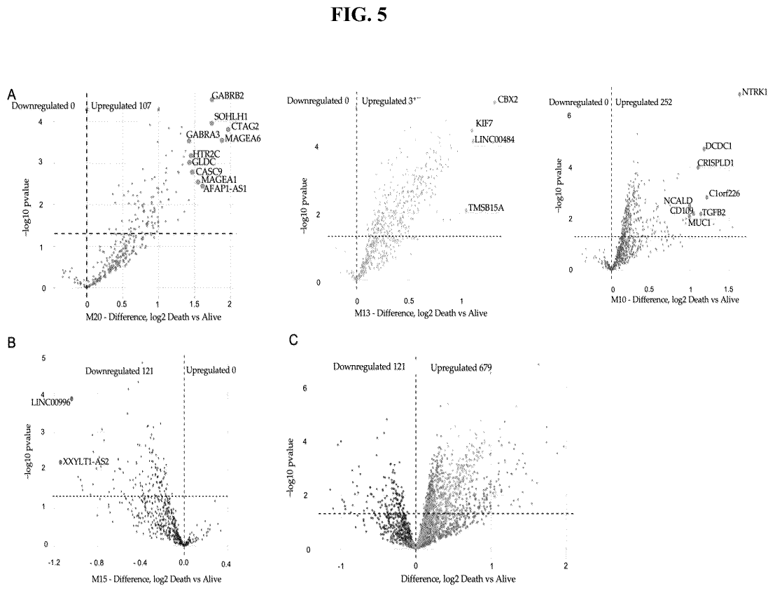

Modules with positive clinical correlations will have high gene expression based on the vital status trait correlation. In contrast, the negatively correlated modules will have low expression in MM patients who died while on RVD treatment. T-test analysis was used to test this hypothesis. Tukey post hoc confirms elevated expression of modules (M10, M13, M20) and lower expression (M15), which were viewed via Volcano plots (FIG. 5). A total of 121 genes were downregulated, and 676 genes were upregulated when the dead and alive MM patients were compared. DEGs from positively correlated significant modules with the highest log 2 fold change values were selected as genes of interest, and these genes were: CTAG, MAGEA6, GABRB6, SOHLH1, NTRK1, AFAP1-AS1, MAGEA1, CCND2, CASC9, HTR2C, GLDC, GABRA3.

TABLE 5

| MM | | | | | | | |
|---|---|---|---|---|---|---|---|
| cleanDat | | | | | | | |
| ANOVA | | | | | | | |
| pairwise | | | | | log2(FPKM) | | |
| comparison | | | | Tukey | differences | | |
| results | | ANOVA | | p Values | Diff | WCGNA | Fold |
| UniqueID | ENSID | F-Value | FDR (BH) | Death-alive | Death-alive | NETcolors | Change |
| MAGEA3 | ENSG00000221867 | 7.17251629 | 0.21903534 | 7.86E−03 | 1.421920852 | royalblue | 2.6794202 |
| NES | ENSG00000132688 | 4.91043512 | 0.35533606 | 0.027535207 | 1.333793001 | purple | 2.60953545 |
| SLCO1A2 | ENSG00000084453 | 12.3732287 | 0.08323217 | 5.11E−04 | 1.374807279 | royalblue | 2.59333266 |
| BCHE | ENSG00000114200 | 6.00914384 | 0.27697497 | 1.49E−02 | 1.360671677 | royalblue | 2.56804713 |
| MYO18B | ENSG00000133454 | 11.7433598 | 0.09517333 | 7.07E−04 | 1.349971765 | royalblue | 2.54907137 |
| CBX2 | ENSG00000173894 | 21.7114638 | 0.01529222 | 5.00E−06 | 1.337036695 | salmon | 2.52631878 |
| SEMA3A | ENSG00000075213 | 10.8854256 | 0.110404 | 1.10E−03 | 1.334233499 | royalblue | 2.52141484 |
| STK32A | ENSG00000169302 | 14.9728776 | 0.06518483 | 1.37E−04 | 1.322126261 | royalblue | 2.50034342 |
| SEMA3D | ENSG00000153993 | 14.2531278 | 0.07215924 | 1.97E−04 | 1.307103234 | royalblue | 2.47444201 |
| PKP2 | ENSG00000057294 | 5.82230782 | 0.28915181 | 0.016495723 | 1.302882453 | purple | 2.46721331 |
| CD3E | ENSG00000198851 | 10.0395157 | 0.1322138 | 1.71E−03 | 1.28093573 | royalblue | 2.42996533 |
| PAGE1 | ENSG00000068985 | 5.20329166 | 0.33528552 | 2.33E−02 | 1.270833973 | royalblue | 2.41301013 |
| DPY19L2 | ENSG00000177990 | 10.1336826 | 0.13036996 | 1.63E−03 | 1.270674332 | royalblue | 2.41274314 |
| LINC01287 | ENSG00000234722 | 8.00170281 | 0.18657923 | 5.03E−03 | 1.243924014 | royalblue | 2.36841848 |
| C1orf226 | ENSG00000239887 | 10.3334308 | 0.12808308 | 0.001466409 | 1.216897327 | purple | 2.32446278 |
| AACSP1 | ENSG00000250420 | 12.3702421 | 0.08323217 | 5.12E−04 | 1.186247681 | royalblue | 2.2756011 |
| DCDC1 | ENSG00000170959 | 18.9452947 | 0.03447327 | 1.92E−05 | 1.181925251 | purple | 2.26879342 |
| POU6F2 | ENSG00000106536 | 14.8486415 | 0.06518483 | 1.46E−04 | 1.143021578 | royalblue | 2.20843073 |
| MM | | | | | | | |
| cleanDat | | | | | | | |
| ANOVA | | | | | | | |
| pairwise | | | | | Log2(FPKM) | | |
| comparison | | | | Tukey | differences | | |
| results | | ANOVA | | p Values | Diff | WCGNA | Fold |
| UniqueID | ENSID | F-Value | FDR (BH) | Death-alive | Death-alive | NETcolors | Change |
| CTAG2 | ENSG00000126890 | 14.6939923 | 0.06518483 | 1.58E−04 | 1.969112125 | royalblue | 3.91527088 |
| MAGEA6 | ENSG00000197172 | 13.5133196 | 0.07513653 | 2.86E−04 | 1.88310824 | royalblue | 3.68868921 |
| GABRB2 | ENSG00000145864 | 18.0265463 | 0.03832796 | 3.01E−05 | 1.743579786 | royalblue | 3.34865045 |
| SOHLH1 | ENSG00000165643 | 15.3802021 | 0.06331129 | 1.12E−04 | 1.741157299 | royalblue | 3.34303231 |
| NTRK1 | ENSG00000198400 | 29.2401024 | 0.00433238 | 1.42E−07 | 1.634923963 | purple | 3.10571181 |
| AFAP1-AS1 | ENSG00000272620 | 8.61147695 | 0.16403257 | 3.63E−03 | 1.614253562 | royalblue | 3.06153158 |
| MAGEA1 | ENSG00000198681 | 9.03799594 | 0.15379295 | 2.90E−03 | 1.55100075 | royalblue | 2.93020327 |
| CCND2 | ENSG00000118971 | 5.37464946 | 0.32312977 | 0.021184336 | 1.544469098 | purple | 2.91696707 |
| CASC9 | ENSG00000249395 | 10.0888844 | 0.13216723 | 1.67E−03 | 1.469280973 | royalblue | 2.76883863 |
| HTR2C | ENSG00000147246 | 11.8591168 | 0.09386565 | 6.66E−04 | 1.452338658 | royalblue | 2.7365129 |
| GLDC | ENSG00000178445 | 11.1151975 | 0.10359968 | 9.77E−04 | 1.429787007 | royalblue | 2.69406938 |
| GABRA3 | ENSG00000011677 | 13.4449923 | 0.07513653 | 2.96E−04 | 1.424029304 | royalblue | 2.68333895 |

The biological functions associated with upregulated genes in the shaded module are skeletal system morphogenesis, G protein-coupled receptor (GPCR) protein signaling pathways, multicellular organismal development, synaptic transmission, and cell-cell adhesion. It was observed that some of the top differentially expressed genes are known MM biomarkers: CTAG2, MAGEA6, MAGEA1, and SSX1. Whereas some genes within the M20 module were differentially expressed, but not well known in the context of MM biology (SOHLH1, GABRA3, GABRB2, HTR2C, and GLDC). It was also noted the top differentially expressed genes in each vital status-associated module based on (−log 10(p-value)=2 and Diff log 2=1) (FIG. 5): NTKR1, MUC1, C1orf226, DCDC1, TGFB2, CRISPLD1, CD109 and NCALD (M10). CBX2, LINC00484, KIF7, KIF14, and TMSB158 (M13). CTAG2, MAGEA6, GABRB2, SOHLH1, AFAP1-AS1, MAGEA1, CASC9, HTR2C, GLDC and GABRA3 (M20).

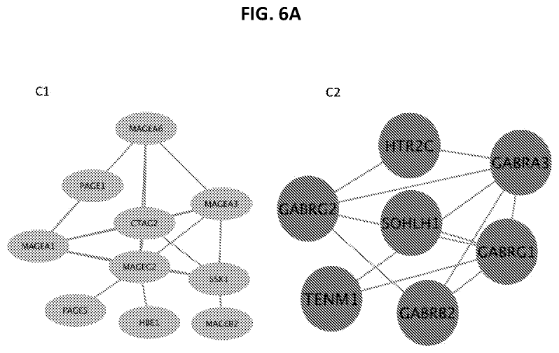

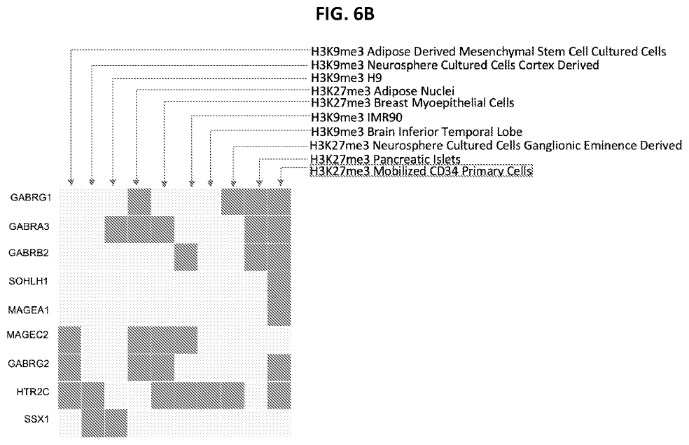

To understand how the genes in M20 are biologically connected in terms of their function, the String database was used. Using the K-means clustering algorithm, differentiated genes in M20 were clustered into two groups (aqua and red nodes) (FIG. 6). Genes denoted with aqua nodes mostly form the MAGE (Melanoma Antigen Gene) family of genes. On the other hand, the gene cluster denoted with red nodes is mostly Gamma-aminobutyric acid receptor genes, which are ligand-gated chloride channels activated by major inhibitory neurotransmitters in the mammalian brain. GABA genes interact with other genes in the M20, such as TENM1. MAGE also interacts with HTR2C, a G-protein coupled receptor and Serotonin receptor. Additionally, MAGE interacts with SOHLH1, a male and female germline differentiation transcription regulator. (FIG. 6A).

The String database was used to analyze Protein to Protein Interaction and other key characteristics for candidate proteins—GABRA3 and BDKRB1 in M20. String-db (v11) was used to score, analyze, and visualize the interaction networks between GABA receptor and BDKRB1 via inflammatory pathways. The PPI analysis predicted new biological functions for GABRA3 and BDKRB1, involving direct or indirect interactions, based on variable confidence levels, evidence-based criteria, and modes of action. PPI analysis predicted biological and molecular functions such as Gaq signaling, GPCR signaling, inflammatory response, and transmembrane signaling receptor activity for interaction between GABRA3 and BDKRB1.

### Example 4: Gene Set Enrichment, ROC Curve, and Kaplan-Meier Analyses

To summarize the differentially expressed genes' biological function and enable further downstream analysis between the different gene sets cluster from the string database, Enrichr was used. Enricir can assign the genes their GO terms, which it retrieves from more than 192 gene set libraries (https://maayanlab.cloud/Enrichr/). The tool mapped most of the significant differentially expressed genes (GABRB2, SOHLH1, MAGEA, GABRA3, HTR2C, GABRG1, and GABRG2) to mobilized CD34 primary cells with a p-value of 0.02 and odds ratio of 3.5.

TABLE 6

| | | | Odds | Combined | |
|---|---|---|---|---|---|
| Term | Overlap | P-value | Ratio | Score | Genes |
| H3K9me3 Adipose Derived | 3/247 | 6.12E−04 | 22.0663189 | 163.247554 | HTR2C; MAGEC2; |
| Mesenchymal Stem Cell | | | | | GABRG2 |
| Cultured Cells | | | | | |
| H3K9me3 Neurosphere | 2/340 | 0.02291286 | 9.68836292 | 36.583811 | HTR2C; SSX1 |
| Cultured Cells Cortex | | | | | |
| Derived | | | | | |
| H3K9me3 H9 | 2/344 | 0.02341863 | 9.57309942 | 35.9395531 | GABRA3; SSX1 |
| H3K27me3 Adipose Nuclei | 4/1174 | 0.00735641 | 6.43282051 | 31.5991908 | GABRA3; MAGEC2; |
| | | | | | GABRG2; GABRG1 |
| H3K27me3 Breast | 4/1292 | 0.01028346 | 5.8068323 | 26.5791401 | GABRA3; HTR2C; |
| Myoepithelial Cells | | | | | MAGEC2; GABRG2 |
| H3K9me3 IMR90 | 3/814 | 0.01747222 | 6.44826813 | 26.097062 | GABRB2; HTR2C; |
| | | | | | MAGEC2 |
| H3K27me3 Mobilized CD34 | 7/53 | 0.02081519 | 3.49527665 | 13.5339638 | GABRB2; SOHLH1; |
| Primary Cells | | | | | GABRA3; MAGEA1; |
| | | | | | HTR2C; GAGRG2; |
| | | | | | GABRG1 |

CD34 is expressed on hematopoietic stem cells and non-hematopoietic cells (mesenchymal stem cells, endothelial cells, etc.). CD34 (+) cells frequently undergo cellular division and formed rapid colonies. Genes associated with CD34 (hematopoietic stem cell) mobilization can be used to predict poor outcomes in MM (FIG. 6B).

To identify specific genes associated with poor outcomes in MM, Receiver Operating Characteristic (ROC) analysis was conducted. Among the gene list, NTRK1 (0.71), GABRB32 (0.67), SOHLH1 (0.65), GABRA3 (0.64), DCDC1 (0.64), MAGEA (0.63), and HTR2C (0.63) have the highest AUC scores of predicting poor outcomes in MM (Table 7).

TABLE 7
ROC AUC scores and p-values for the top 10 genes
based on their log fold change values.

| Module Name | Gene | AUC | z | p-value |
|---|---|---|---|---|
| Royalblue | CTAG2 | 0.64854 | 3.5529 | 0.00038 |
| | MAGEA6 | 0.63382 | 3.0658 | 0.00217 |
| | GABRB2 | 0.66859 | 4.19179 | 3.00E−05 |
| | SOHLH1 | 0.64846 | 3.47324 | 0.00051 |
| | AFAP1_AS1 | 0.62386 | 3.05481 | 0.00225 |
| | MAGEA1 | 0.62911 | 3.14134 | 0.00168 |
| | CASC9 | 0.61675 | 2.63932 | 0.00831 |
| | HTR2C | 0.6261 | 2.87452 | 0.00405 |
| | GLDC | 0.63476 | 3.31424 | 0.00092 |
| | GABRA3 | 0.63484 | 3.15689 | 0.00159 |

The input genes had AUC scores above 0.6. GABRB2 (0.669), CTAG2 (0.649), SOHLH1 (0.648), GLDC (0.635) and GABRA3 (0.635) have the highest AUC scores.

At the same time, GABRG1 (0.57) and GABRG2 (0.60) have the lowest AUC scores for predicting poor outcomes in MM. To improve the predictive ability of AUC, the AUCs of the highest predictive scores were combined, and there were no significant differences. The ROC result from the MMRF dataset shows that the genes of interest from the royalblue module are not promising MM biomarkers due to their low AUC values, but they have significant p-values. To validate this result, a microarray dataset of 602 individuals with MM disease from the Gene Expression Omnibus (GLO) database (GSE83503) was downloaded. The dataset was log 2 transformed before the ROC analysis and processed similarly as the MMRF dataset. The AUC scores from GLO dataset aligned with that of the MMRF CoMMpass database (Table 8A and 8B).

TABLE 8A
MMRF Compass Dataset

| Marker | AUC | SE.AUC | Lower Limit | Upper Limit | z | p-value |
|---|---|---|---|---|---|---|
| GABRA3 | 0.63484 | 0.04271 | 0.55112 | 0.71856 | 3.15689 | 0.00159 |
| CTAG2 | 0.64854 | 0.04181 | 0.5666 | 0.73049 | 3.5529 | 0.00038 |
| MAGEA1 | 0.62911 | 0.0411 | 0.54855 | 0.70966 | 3.14134 | 0.00168 |
| GABRB2 | 0.66859 | 0.04022 | 0.58976 | 0.74742 | 4.19179 | 3.00E−05 |
| GLDC | 0.63476 | 0.04066 | 0.55507 | 0.71445 | 3.31424 | 0.00092 |

TABLE 8B
GSE83503 Micro-Array Dataset

| Marker | AUC | SE.AUC | Lower Limit | Upper Limit | z | p-value |
|---|---|---|---|---|---|---|
| GABRA3 | 0.61784 | 0.02629 | 0.56631 | 0.66937 | 4.48192 | 1.00E−05 |
| CTAG2 | 0.52357 | 0.02805 | 0.4686 | 0.57854 | 0.84033 | 0.40072 |
| MAGEA1 | 0.5294 | 0.02717 | 0.47614 | 0.58267 | 1.08208 | 0.27922 |
| GABRB2 | 0.59936 | 0.02688 | 0.54668 | 0.65205 | 3.69628 | 0.00022 |
| GLDC | 0.5228 | 0.02749 | 0.46893 | 0.57667 | 0.82943 | 0.40686 |

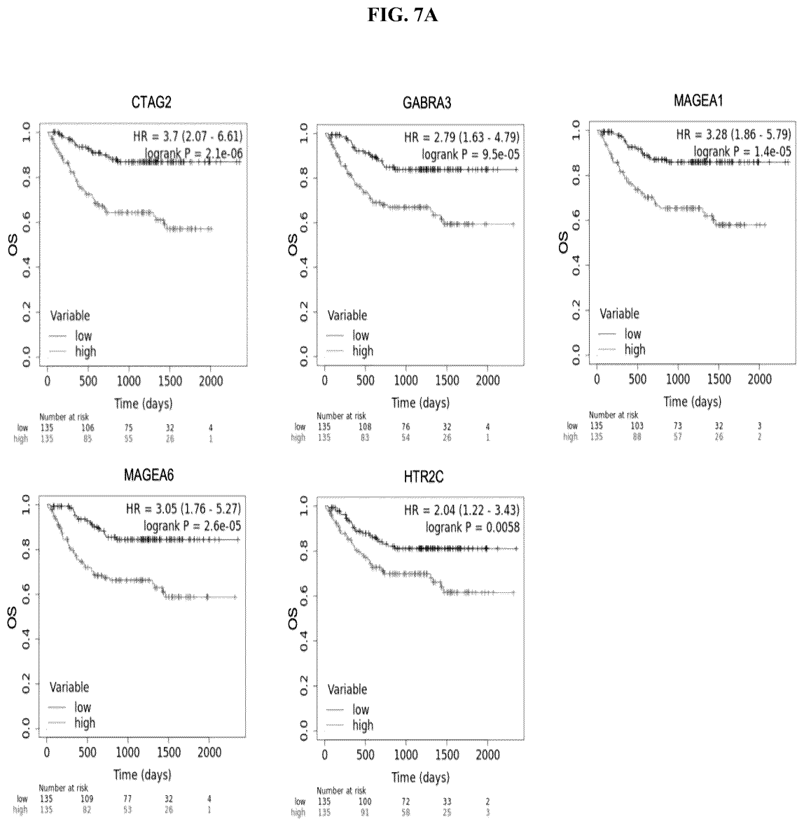

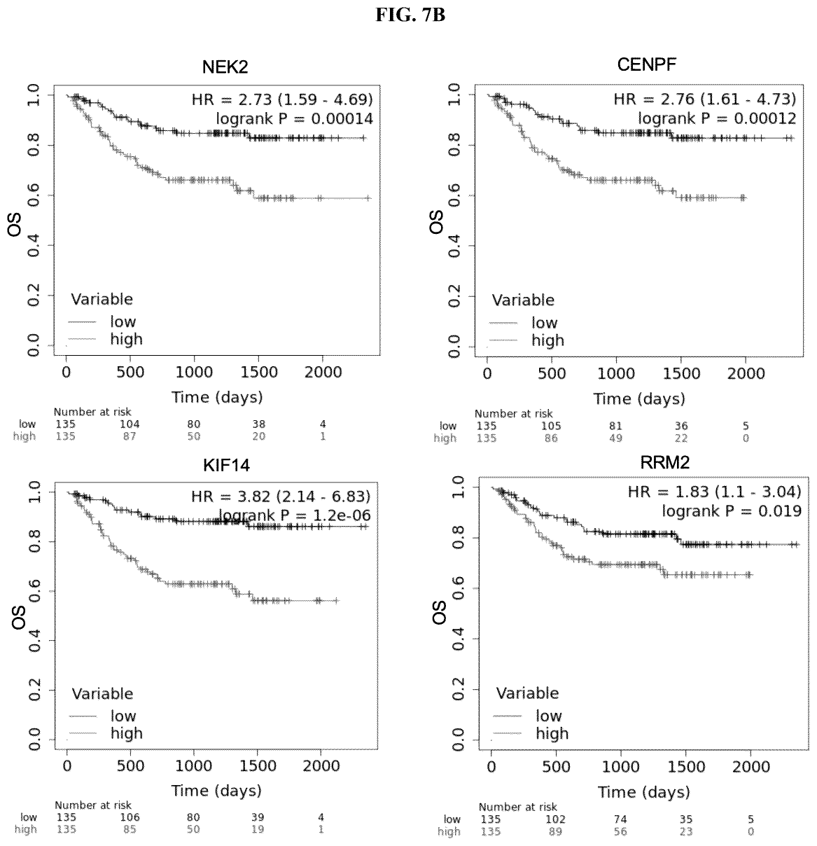

To determine the effect of differentially expressed hub genes on MM overall survival outcomes, a log-rank Kaplan-Meier (KM) analysis using KM plotter was performed (FIG. 7A). Hub genes were selected for this analysis as they would make good therapeutic candidates due to their ability to regulate multiple gene expressions. Differentially expressed hub genes (p-value <0.05, kME >0.7, Log Fold Change (LFC) ≥0.5) from the modules positively correlated with MM4 vital status (M10, M13 and M20) were evaluated (Table 9; FIG. 7B).

TABLE 9

| UniqueID | F-Value | FDR(BH) | P-value | NETcolors | Fold change | kME |
|---|---|---|---|---|---|---|
| NEK2 | 14.8821512 | 0.06518483 | 0.00014341 | salmon | 1.96988507 | 0.88964613 |
| KIF4 | 17.1945878 | 0.04201811 | 4.53E−05 | salmon | 1.87196404 | 0.88515559 |
| CENPF | 15.7973229 | 0.05782571 | 9.07E−05 | salmon | 1.87982766 | 0.87578245 |
| GABRA3 | 13.4449923 | 0.07513653 | 2.96E−04 | royalblue | 2.68333895 | 0.80449542 |
| RRM2 | 15.3072578 | 0.15379295 | 0.00011589 | salmon | 1.88772793 | 0.79906078 |
| MAGEA6 | 13.5133196 | 0.09386565 | 2.86E−04 | royalblue | 3.68868921 | 0.77976914 |
| MAGEA1 | 9.03799594 | 0.06518483 | 2.9E−03 | royalblue | 2.93020327 | 0.7200529 |
| HTR2C | 11.8591168 | 0.09386565 | 6.66E−04 | royalblue | 2.7365129 | 0.71580957 |
| CTAG2 | 14.6939923 | 0.06518483 | 1.58E−04 | royalblue | 3.91527088 | 0.70009416 |
| SOHLH1 | 15.3802021 | 0.06331129 | 1.12E−04 | royalblue | 3.34303231 | 0.6762739 |
| FAM72C | 13.782331 | 0.07410552 | 0.00024966 | salmon | 1.85106907 | 0.65980026 |
| CASC9 | 10.0888844 | 0.13216723 | 1.67E−03 | royalblue | 2.76883863 | 0.64651398 |
| NES | 4.91043512 | 0.35533606 | 0.02753521 | purple | 2.60953545 | 0.61447718 |
| AFAP1-AS1 | 8.61147695 | 0.16403257 | 3.63E−03 | royalblue | 3.06153157 | 0.60704947 |
| CCND2 | 5.37464945 | 0.32312977 | 0.02118434 | purple | 2.91696707 | 0.58697277 |
| CCD109 | 7.5785211 | 0.20228779 | 0.00631043 | purple | 2.06580113 | 0.49279116 |
| RNU6-583P | 5.8954236 | 0.28371926 | 0.01583895 | salmon | 1.87731759 | 0.48424885 |
| KIF7 | 17.7334367 | 0.0392808 | 3.47E−05 | salmon | 2.16963657 | 0.46775778 |
| NTRK1 | 29.2401024 | 0.00433237 | 1.42E−07 | purple | 3.10571181 | 0.46018116 |
| PKP2 | 5.82230782 | 0.28915181 | 0.01649572 | purple | 2.46721331 | 0.44459126 |
| TMSB15A | 7.086524 | 0.22320869 | 0.00823592 | salmon | 2.0862709 | 0.3793585 |
| CBX2 | 21.7114638 | 0.01529222 | 5.00E−06 | salmon | 2.52631878 | 0.37773077 |
| LINC00484 | 16.2865821 | 0.05058776 | 7.11E−05 | salmon | 2.18544446 | 0.35595025 |

KM analysis showed significant difference in the OS of patients with low vs. high expression of our genes of interest. However, MiG did not satisfy the cutoff points. Additionally, KM plotter was used to calculate the Pearson correlation between the genes in M20 and M13, respectively. MAGEA6 and GABRA3 in M20 have a positive linear correlation of 90%, while KIF14 and CENPF in M13 have a positive linear correlation of 94% (Table 10A and 10B).

TABLE 10A

| Affymetrix | | | | | |
|---|---|---|---|---|---|
| ID | CTAG2 | GABRA3 | MAGEA1 | MAGEA6 | HTR2C |
| CTAG2 | 1 (p < 1E−04) | | | | |
| GABRA3 | 0.6552 (p < 1E−04) | 1 (p < 1E−04) | | | |
| MAGEA1 | 0.6456 (p < 1E−04) | 0.6289 (p < 1E−04) | 1 (p < 1E−04) | | |
| MAGEA6 | 0.6363 (p < 1E−04) | 04) 0.9012 (p < 1E−04) | 0.5964 (p < 1E−04) | 1 (p < 1E−04) | |
| HTR2C | 0.5693 (p < 1E−04) | 0.5967 (p < 1E−04) | 0.5939 (p < 1E−04) | 0.5106 (p < 1E−04) | 1 (p < 1E−04) |

TABLE 10B

| Affymetrix ID | NEK2 | CENPF | KIF14 | RRM2 |
|---|---|---|---|---|
| NEK2 | 1 (p < 1E−04) | | | |
| CENPF | 0.9176 (p < 1E−04) | 1 (p < 1E−04) | | |
| KIF14 | 0.9145 (p < 1E−04) | 0.9352 (p < 1E−04) | 1 (p < 1E−04) | |
| RRM2 | 0.7796 (p < 1E−04) | 0.8067 (p < 1E−04) | 0.8115 (p < 1E−04) | 1 (p < 1E−04) |

### Example 5: Diagnostic and Prognostic Values of Hub Genes in MM

First, the ROC curve analysis was performed among 5 hub genes based on the expression values from M20. M20 was selected for further analysis because of its top biological functions associated with genes in this module: MAGEA3, VN1R54P, GABRA3, BDKRB1 and GABRG2. Because prediction was based on right censored data, a COX model was constructed for ROC curve for survival data, and the following results were obtained at different times, where GABRA3 predictive accuracy ranged from 60-80% while BDKRB1 predictive accuracy ranged from 60-78% with t ranged from 0-2000).

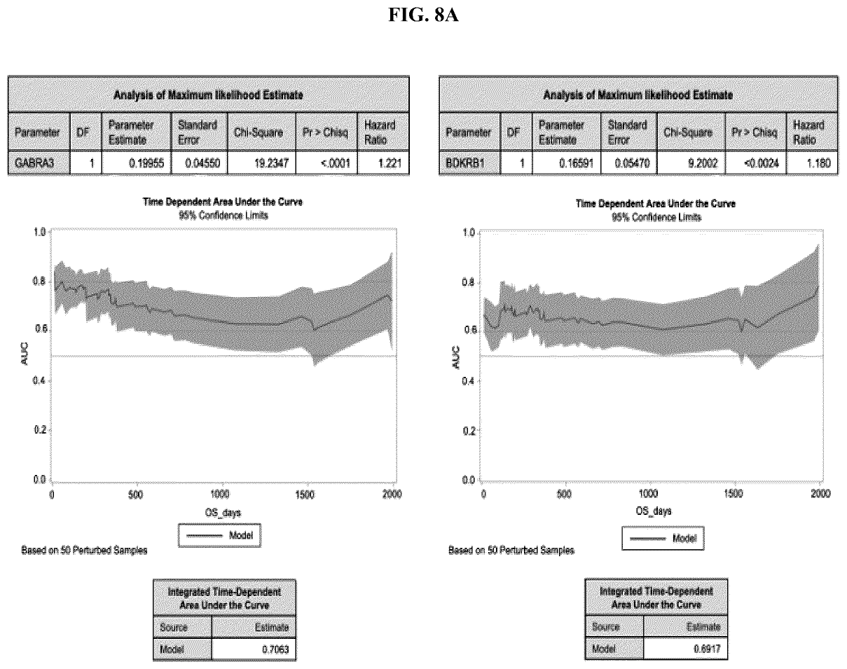

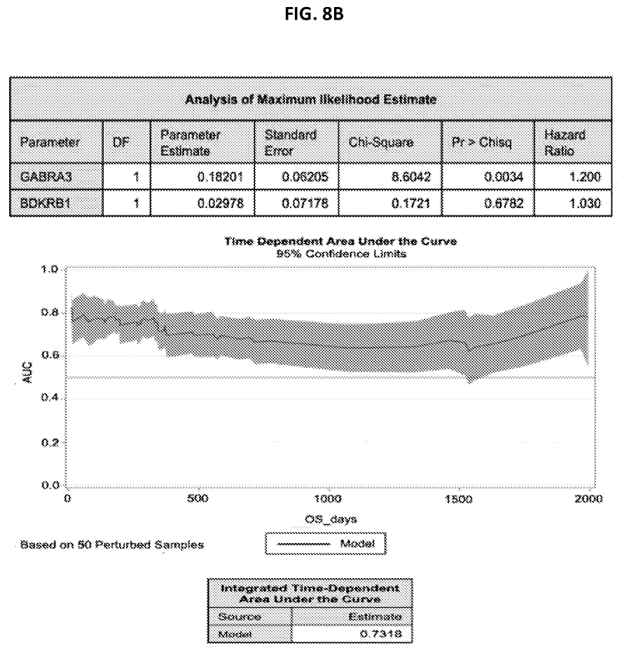

A SAS COX model was used to construct time-dependent AUC values of 6900 for BDKRB1 and 70% for GABRA3 for prognostic assessment (FIG. 8A). The combination of BDKRB 1 and GABRA3 AUC fairly improved the diagnostic performance of the biomarker in predicting MM mortality (AUC=73%) (FIG. 8B). Additionally, ROC curve analysis was performed on the top 5 differentially expressed genes (DEGs) (positively correlated modules (M20, M13, and M10) among MM patients who died while on RVD versus those who are alive. These genes include CTAG2, MAGEA6, SOHLH1, MAGEA1, and AFAP1-AS1 in M20; CBX2, LINC00484, KIF7, TMSB15A, and NEK2 in M13; and NTRK1, CCND2, NES, PKP2, and C1orf226 in M10. Of note, NEK2 in M13 and CCND2 in M10 are bona fide myeloma biomarkers. The top 5 genes in the negatively correlated module M15 (IFITM1, CDH23, AGRN, DHX58, and LINC02576) were associated with positive RVD treatment outcomes in MM patients.

### Example 6: Gene Expression and Clinical Outcome Association in MM Vital Status

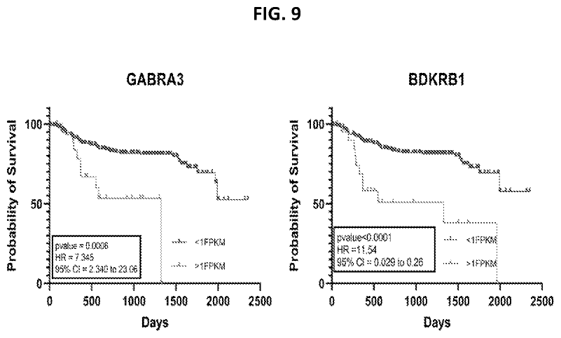

Overall survival (OS), defined as the duration of survival after a disease is diagnosed or treated, is one of the gold standard parameters for assessing clinical outcomes or endpoints based on the patient's vital status (death). To further identify the hub genes and the DEG biomarkers that are associated with OS, a Log-ranked Kaplan-Meier analysis was performed (FIG. 9). Hub gene biomarkers (GABRA3, and BDKRB1) were selected for this analysis since they have the potential to predict MM vita status.

Since BDKRB1 and GABRA3 are only expressed in a subset of patients stratifying by median or mean expression level would group many patients that do not express these genes in the high expression category. Thus, a cutoff of 1 FPKM was chosen as this has been shown to correspond with approximately 1 mRNA/cell in plasma cells [Barwick, B. G., et al., Plasma cell differentiation is coupled to division-dependent DNA hypomethylation and gene regulation. Nat Immunol, 2016. 17(10): p. 1216-1225]. That being said, to ensure the validity of results thresholds of 0.5 FPKM were used, and this data show that BDKRB1 and GABRA3 expression are still associated with worse outcomes.

Two key hub genes (GABRA3, and BDKRB1) have been identified based on their KME values (GABRA3 (KME: 0.8045), and BDKRB1(KME: 0.7928)). GABRA3 is one of the first five highly expressed genes in M20, and one of the GABA receptor subunits that controls the inhibitory signaling of neurotransmitters in the central nervous system. Cancer cells take advantage of the neurotransmitters-initiated signaling pathway to activate uncontrolled proliferation and dissemination. In addition, neurotransmitters can also affect immune cells and endothelial cells in the tumor microenvironment to promote tumor progression. BDKRB1 is also one of the first 5 highly expressed genes in M20. This gene product supports calcium influx, with subsequent activation of the MEK1-ERK1/2-NF-κB pathway leading to cancer progression. The expression of both GABRA3 and BDKRB1 genes was found in a certain subset of MM patients having a significantly higher incidence of deaths due to MM. Therefore, they can serve as therapeutic targets in MM patients.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended unless the context specifically indicates the contrary.

What is claimed is:

1. A method of diagnosing and treating multiple myeloma (MM) in a subject comprising,
(a) measuring the level of one or more biomarkers in a sample from the subject;
(b) comparing the level of the one or more biomarkers to a reference level of the one or more biomarkers;
(c) making a diagnosis based on the result of the comparing step; and
(d) treating the subject with one or more active agents where the subject is diagnosed with multiple myeloma.

2. The method of claim 1, comprising measuring the levels of at least 5 MM biomarkers,
wherein the biomarkers are selected from one or more of GABRA3, CTAG2, MAGEA6, SOHLH1, MAGEA1, AFAP1-AS1; CBX2, LINC00484, KIF7, TMSB15A, NEK2, NTRK1, CCND2, NES, PKP2, and C1orf226, and
wherein increased activity or expression of the MM biomarker in the sample as compared to a control is indicative of multiple myeloma.

3. The method of claim 1, comprising measuring the levels of at least 5 MM biomarkers, wherein the biomarkers are selected from one or more of IFITM1, CDH23, AGRN, DHX58, and LINC02576,
wherein decreased activity or expression of the MM biomarker in the sample as compared to a control is indicative of multiple myeloma.

4. The method of claim 1, wherein the biomarker is GABRA3.

5. The method of claim 1, wherein the sample comprises blood plasma cells or bone marrow cells.

6. The method of claim 1, wherein the sample is a urine sample.

7. The method of claim 4, comprising administering a GABAA or GABRA3 receptor antagonist.

8. The method of claim 7, wherein the antagonist is one or more selected from the group consisting of flumazenil, thiocolchicoside, pentetrazol, picrotoxin, topiramate, loreclezole, etomitade and propofol.

9. The method of claim 7, wherein the antagonist is an antibody directed against the GABAA alpha-3 subunit.

10. The method of claim 7, wherein the antagonist is a polynucleotide.

11. The method of claim 10, wherein the polynucleotide is an RNA selected from the group consisting of a short interfering RNA (siRNA), a short hairpin RNA (shRNA) molecule, an antisense RNA, and a microRNA (miRNA).

12. The method of claim 10, wherein the polynucleotide is an A-to-I RNA edited GABRA3 encoded polynucleotide.

13. A method for determining disease progression or risk for metastasis in a subject with multiple myeloma, comprising the steps of:
(a) measuring the level of one or more biomarkers in a first sample obtained from the subject with multiple myeloma at a first time point;
(b) measuring the level of the one or more biomarkers in a second sample obtained from the subject at a second time point;
(c) comparing the level of the one or more biomarkers at the first time point to the level of the one or more biomarkers at the second time point;
(d) determining the disease progression between the first and the second time point based on the result of step (c); and
(e) further treating the subject with one or more active agents if the multiple myeloma has progressed.

14. A method for determining the efficacy of a treatment for multiple myeloma in a subject, comprising the steps of:
(a) measuring the level of one or more biomarkers in a first sample obtained from the subject at a first time point;
(b) measuring the level of the one or more biomarkers in a second sample obtained from the subject at a second time point, wherein the subject is under treatment at the second time point;
(c) comparing the level of the one or more biomarkers at the first time point to the level of the one or more biomarkers at the second time point;
(d) determining the efficacy of the treatment based on the result of step (c); and
(e) further treating the subject with one or more active agents if the efficacy has been found to be insufficient for treatment. In certain preferred embodiments, the one or more active agents in step (e) include one or more active agents that were not administered in the previous treatment.

15. The method of diagnosing and treating of claim 1, wherein the treatment comprises administering to a subject in need thereof, one or more agents increasing or inhibiting the expression or activity of one or more MM biomarkers in amounts sufficient to inhibit the progression of multiple myeloma in the subject,
wherein the one or more MM biomarkers correspond to gene products selected from one or more of GABRA3, CTAG2, MAGEA6, SOHLH1, MAGEA1, AFAP1-AS1; CBX2, LINC00484, KIF7, TMSB15A, NEK2, NTRK1, CCND2, NES, PKP2, C1orf226, IFITM1, CDH23, AGRN, DHX58, and LINC02576.

16. The method of claim 15, wherein the one or more agents inhibit the expression or activity of biomarker marker identified from one or more of GABRA3, CTAG2, MAGEA6, SOHLH1, MAGEA1, AFAP1-AS1; CBX2, LINC00484, KIF7, TMSB15A, NEK2, NTRK1, CCND2, NES, PKP2, and C1orf226.

17. The method of claim 15, wherein the one or more agents increase the expression or activity of biomarker marker selected from one or more of IFITM1, CDH23, AGRN, DHX58, and LINC02576.

18. The method of claim 16, wherein the one or more agents comprise a small molecule GABAA or GABRA3 receptor antagonist selected from the group consisting of flumazenil, thiocolchicoside, pentetrazol, picrotoxin, topiramate, loreclezole, etomitade and propofol.

19. The method of claim 16, wherein the one or more agents comprise an antibody directed against the GABAA alpha-3 subunit.

20. The method of claim 13, wherein the one or more agents comprise an RNA selected from the group consisting of short interfering RNA (siRNA), short hairpin RNA (shRNA) molecule, antisense RNA, and microRNA (miRNA).
